(19) United States
(12) Patent Application Publication
(10) Pub. No.: US 2022/0175833 A1
(43) Pub. Date: Jun. 9, 2022

(54) TARGETED DELIVERY OF IMMUNE-MODULATING VHH AND VHH-FUSION PROTEIN

(71) Applicants: Children's Medical Center Corporation, Boston, MA (US); Massachusetts Institute of Technology, Cambridge, MA (US)

(72) Inventors: Yushu J. Xie, Brookline, MA (US); Hidde L. Ploegh, Boston, MA (US)

(73) Assignees: Children's Medical Center Corporation, Boston, MA (US); Massachusetts Institute of Technology, Cambridge, MA (US)

(21) Appl. No.: 17/436,131

(22) PCT Filed: Mar. 6, 2020
(86) PCT No.: PCT/US2020/021466

§ 371 Date: Sep. 3, 2021

Related U.S. Application Data
(60) Provisional application No. 62/815,318, filed on Mar. 7, 2019.

(51) Int. Cl. A61K 35/17; C07K 16/28

(52) U.S. Cl. CPC A61K 35/17; C07K 16/2827; C07K 2319/33; C07K 2317/569; C07K 2317/22

(57) ABSTRACT

Provided herein are engineered cells that comprising a chimeric antigen receptor comprising an extracellular target-binding moiety and an intracellular signaling domain; and secrets a heavy-chain antibody (VHH) or a VHH fusion protein. Methods of using the engineered cell to treat a disease (e.g., cancer or autoimmune disease) are also provided.

### RELATED APPLICATIONS

This application claims the benefit under 35 U.S.C. § 119(e) to U.S. Provisional Application No. 62/815,318, filed Mar. 7, 2019, entitled “TARGETED DELIVERY OF IMMUNE-MODULATING VHH AND VHH-FUSIONS USING CELLULAR THERAPIES,” the entire contents of which are incorporated herein by reference.

### GOVERNMENT SUPPORT

This invention was made with government support under the Graduate Research Fellowship Program (GRFP), awarded by the National Science Foundation. The government has certain rights in this invention.

### BACKGROUND

Engineered T cells expressing chimeric antigen receptors (CAR-T cells) targeting tumor specific antigens have been adopted in the clinic for cancer immunotherapy. Very few tumor specific antigens have been found for solid tumors. Solid tumors also have a highly immunosuppressive microenvironment.

### SUMMARY

The present disclosure, in some aspects, provides engineered cells (e.g., engineered immune cells) that express a chimeric antigen receptor (CAR) and is capable of enhancing tumor killing (e.g., when used in cancer immunotherapy) by secreting immune-modulating VHHs or VHH-fusion proteins. In some embodiments, the VHH-secreting CARs described herein are used for the treatment of auto-immune disease, by targeting the VHH secreting CAR T cells to over-reactive cells such that they are removed from circulation.

As described herein, the engineered cells comprising a chimeric antigen receptor (e.g., CAR-T cells) can be used as delivery vehicles for localized expression of immune-modulating VHHs or VHH-fusion proteins. Due to its production by the engineered cells, the VHHs or VHH-fusion proteins provide a self-renewing source of therapeutics, avoiding potential toxicities caused by systemic injection of immune-modulating molecules (e.g., checkpoint-blocking molecules) and also eliminating the need for constant antibody dosing, as the engineered cells themselves are capable of producing therapeutics.

Further, the small size, high stability and solubility of VHHs render them superior to monoclonal antibodies, sc-Fvs, or similar variants as secreted immune-modulating molecules. Since secondary folding is not required for VHHs, they can generally be produced stably with high expression and low metabolic strain on the cell. In some embodiments, the VHHs are further combined into dimers or fused with additional moieties, such as an Fc domain, for additional functionalities. With VHH Fc-fusion-secreting CAR cells (e.g., CAR T cells), “anti-body-like” molecules with effector functions are localized to a certain target, potentially increasing the safety profile of the therapeutic strategies.

Accordingly, some aspects of the present disclosure provide engineered cells comprising a chimeric antigen receptor (CAR) comprising an extracellular target-binding moiety and an intracellular signaling domain, wherein the engineered cell secretes a VHH or a VHH fusion protein.

In some embodiments, the engineered cell comprises: (i) a nucleotide sequence encoding a chimeric antigen receptor (CAR) comprising an extracellular target-binding moiety and an intracellular signaling domain; and (ii) a nucleotide sequence encoding a heavy-chain antibody (VHH) or a VHH fusion protein thereof. In some embodiments, the nucleotide sequence of (i) is operably linked to a first promoter. In some embodiments, the engineered cell secretes the VHH or VHH fusion protein.

In some embodiments, the nucleotide sequence of (i) and/or (ii) is operably linked at the 5′ end to a nucleotide sequence encoding a signal sequence. In some embodiments, (i) and (ii) are linked via a nucleotide sequence encoding a self-cleaving peptide. In some embodiments, the self-cleaving peptide is a P2A peptide. In some embodiments, (i) and (ii) are linked via an internal ribosome entry site (IRES). In some embodiments, (ii) is operably linked to a second promoter. In some embodiments, (i) and (ii) are on the same vector. In some embodiments, the vector is a lentiviral vector or a retroviral vector.

In some embodiments, the extracellular target-binding moiety of the CAR is an antibody. In some embodiments, the antibody is a full-length antibody, an antigen-binding fragment, a single domain antibody, a single-chain variable fragment (scFv), or a diabody. In some embodiments, the antibody is a single domain antibody. In some embodiments, the single domain antibody is a VHH.

In some embodiments, the extracellular target-binding moiety of the CAR binds a tumor-associated antigen. In some embodiments, the tumor associated antigen is selected from the group consisting of: PDL1, EIIIB fibronectin, CEA, PSMA, AXL, HER2, CD133, Muc1, Muc16, Siglec15, and mesothelin.

In some embodiments, the extracellular target-binding moiety of the CAR binds an autoimmune antigen. In some embodiments, the autoimmune antigen is selected from the group consisting of: antigen-specific T-cell receptors, B cell receptors, and insulin receptor.

In some embodiments, the nucleotide sequence of (ii) encodes a VHH. In some embodiments, the nucleotide sequence of (ii) encodes a VHH fusion protein. In some embodiments, the VHH fusion protein comprises a VHH fused to a fragment crystallizable region (Fc). In some embodiments, the VHH fusion protein comprises a VHH fused to an enzyme, a cytokine, or a different VHH.

In some embodiments, the VHH or VHH fusion protein binds an immune checkpoint protein, a tumor-associated antigen, or an immune cell associated antigen. In some embodiments, the VHH or VHH fusion protein binds a protein selected from the group consisting of: CD47, CTLA4, PD1, PDL1, TIM3, EIIIB fibronectin, LAG3, VISTA, Siglec15, VEGF, VEGFR, HER2, PSMA, AXL, Muc1, Muc16, MHCI/II.

In some embodiments, the extracellular target-binding moiety of the CAR binds PD-L1 and the VHH or VHH fusion protein binds CD47.

In some embodiments, the extracellular target-binding moiety of the CAR binds PD-L1 and the VHH or VHH fusion protein binds CTLA4.

In some embodiments, the extracellular target-binding moiety of the CAR binds PD-L1 and the VHH or VHH fusion protein binds PD-1.

In some embodiments, the extracellular target-binding moiety of the CAR binds PD-L1 and the VHH or VHH fusion protein binds TIM3.

In some embodiments, the extracellular target-binding moiety of the CAR binds PD-L1 and the VHH or VHH fusion protein binds EIIIB fibronectin.

In some embodiments, the extracellular target-binding moiety of the CAR binds EIIB fibronectin and the VHH or VHH fusion protein binds CD47.

In some embodiments, the extracellular target-binding moiety of the CAR binds EIIB fibronectin and the VHH or VHH fusion protein binds CTLA4.

In some embodiments, the extracellular target-binding moiety of the CAR binds EIIB fibronectin and the VHH or VHH fusion protein binds PD-1.

In some embodiments, the extracellular target-binding moiety of the CAR binds EIIB fibronectin and the VHH or VHH fusion protein binds TIM3.

In some embodiments, the extracellular target-binding moiety of the CAR binds EIIB fibronectin and the VHH or VHH fusion protein binds PD-L1.

In some embodiments, the extracellular target-binding moiety of the CAR binds EIIIB fibronectin and the VHH or VHH fusion protein binds LAG3.

In some embodiments, the extracellular target-binding moiety of the CAR binds EIIIB fibronectin and the VHH or VHH fusion protein binds LAG3 and TIM3.

In some embodiments, the extracellular target-binding moiety of the CAR binds PD-L1 and the VHH or VHH fusion protein binds CD47 and CTLA-4.

In some embodiments, cell is an immune cell. In some embodiments, the immune cell is selected from CD4+ T cells, CD8+ T cells, regulatory T cells (Tregs), Natural Killer T (NKT) cells, and Natural Killer (NK) cells.

Further provided herein are compositions comprising the engineered cell described herein. In some embodiments, the composition further comprises a pharmaceutically-acceptable carrier. Further provided herein are the use of the engineered cell or the composition described herein in treating a disease.

Other aspects of the present disclosure provide methods of treating a disease, the method comprising administering to a subject in need thereof a therapeutically effective amount of the engineered cell or the composition described herein.

In some embodiments, the disease is cancer (e.g., a solid tumor). In some embodiments, the disease is an autoimmune disease.

In some embodiments, the engineered cell or the composition is administered via injection or transfusion.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1A shows cytokine secreting CAR T cells. FIG. 1B shows VHH-secreting CAR T cells.

FIG. 2 shows characteristics of successful immunotherapies.

FIGS. 3A-3C show the mechanism of the anti-phagocytic activity of CD47. The figure is adopted from Sockolosky et al., PNAS May 10, 2016 113 (19) E2646-E2654, incorporated herein by reference.

FIG. 4 is a schematic showing that tumor killing efficiency may be enhanced by engaging the innate immune system.

FIG. 5 shows three constructs that were used to generate a VHH secreted CAR T cell.

FIG. 6 is a schematic showing that A4 secreting CARs block detection of CD47.

FIG. 7 shows the CAR T cells are able to secrete soluble CD47 to sufficiently block the fluorescently labeled anti-CD47 mAb from binding.

FIG. 8 shows anti-HA IP on supernatant of A4-HA secreting CARs.

FIG. 9 shows that engineered cells comprising A4 (anti-CD47) CARs and secreting A12 (anti-PD-L1 VHH) function in vitro on B16 melanoma cells.

FIG. 10 shows an in vivo experiment on A4 secreting CARs.

FIG. 11 shows localized A4-secretion improves A12 CAR (A12 CAR means the chimeric antigen receptor comprises an extracellular targeting moiety that is an A12 VHH, which binds PD-L1) T cell treatment.

FIG. 12 shows an experiment verifying that having the excess metabolic strain of producing the A4 VHH did not affect cell persistence.

FIG. 13 shows CAR T cell expansion is not negatively affected by A4 secretion.

FIG. 14 shows epitope spreading seen with A12A4 (engineered cells comprising A12 CAR and secretes A4) treatment.

FIG. 15 shows an ELIspot assay showing epitope spreading.

FIG. 16 shows a construct that can be used to generate VHH-FC fusions, providing potential effector function.

FIG. 17 shows engineered cells containing a construct encoding A12 CAR linked to A4-Fc with a P2A peptide secrete A4-Fc.

FIG. 18 shows that in an IP on the supernatant of the A4Fc secreting CAR T cells, the A4Fc is expressed and secreted.

FIG. 19 shows A12-A4Fc CAR T cell activity in vitro.

FIG. 20 shows an in vivo experiment on A4-Fc secreting CAR T cells.

FIG. 21 is a schematic showing that targeted A4Fc delivery shows less toxicity.

FIG. 22 shows targeted delivery of A4Fc decreases binding to circulating RBCs.

FIG. 23 shows tumor killing efficacy may be enhanced by preventing T cell exhaustion.

FIG. 24 shows anti-PDL1-secreting CAR T cells can be generated to decrease T cell exhaustion.

FIG. 25 shows A12 secreting CART cells block detection of PD-L1.

FIG. 26 shows engineered cells containing a construct encoding B2 CAR linked to A4-A12 with a P2A peptide can secrete functional A12.

FIG. 27 shows anti-HA IP on supernatant of A12-HA secreting CAR T cells.

FIG. 28 shows A12-secreting CAR T cells show less “exhaustion” during generation in vivo.

FIG. 29 shows an in vivo experiment on A12 secreting CAR T cells.

FIG. 30 shows A12 secretion increases persistence of B2 CAR T cells.

FIG. 31 shows B2 CAR T cells secreting A12 do not significantly increase survival over B2 CAR T cells.

FIG. 32 shows engineered cells comprising B2 CARs and secrets H11Fc.

FIG. 33 shows that the engineered cells shown in FIG. 32 secretes H11Fc.

FIG. 34 shows Hi i-Fc secreting CAR T cells show less “exhaustion” during generation in vivo.

FIG. 35 shows an in vivo experiment using Hi i-Fc secreting CAR T cells.

FIGS. 36A-36B show that constructs containing nucleotide sequence encoding B2 (anti-EIIB) CAR linked with nucleotide encoding A4 (anti-CD47) via an IRES or CMV promoter (pCMV) can secrete functional A4.

FIGS. 37A-37B show that A12 (anti-PD-1) containing CAR linked to H11Fc (anti-CTLA-4) via a P2A peptide can be generated.

FIGS. 38A-38C shows that CAR containing A12 (anti-PD-1) can be engineered to secret multiple agents, such as A4 (anti-CD47) and H11Fc (anti-CTLA-4), and that A4 and H11Fc were secreted from the CAR.

FIG. 39 shows that the CAR depicted in FIG. 38A was able to inhibit tumor growth.

FIG. 40 shows that CARs can be used to target delivery of bi-specific VHHs.

### DETAILED DESCRIPTION OF CERTAIN EMBODIMENTS

The present disclosure, in some aspects, provide engineered cells (e.g., engineered immune cells) that express a chimeric antigen receptor (CAR) and is capable of enhancing tumor killing (e.g., when used in cancer immunotherapy) by secreting immune-modulating VHHs or VHH-fusion proteins. The VHH-secreting cells are used in combination therapeutic strategies, where the CAR is used to target the engineered cell to a target site (e.g., a tumor cell), wherein immune-modulating VHHs or VHH fusion proteins are expressed in a localized fashion. In some embodiments, the VHH-secreting cells described herein are used for the treatment of auto-immune disease treatment, by targeting the VHH secreting CAR T cells to over-reactive cells such that they are removed from circulation.

In some aspects, the therapeutic strategies provided herein uses CAR expressing cells (e.g., CAR-T cells) for localized release of therapeutic molecules (e.g., immune-modulating VHHs or VHH fusion proteins), allowing safe delivery of potentially toxic therapeutics. In some aspects, the combination therapeutic strategies provided herein allow constant, self-renewing source of therapeutics. This is advantageous over CAR-T therapies or immune-modulating therapies alone. For example, systemically dosed immune-modulators often undergo a laborious production and purification process and the process can be avoided by having the CAR expressing cells (e.g., CAR T cells) generate these molecules at the target site in a localized fashion. Furthermore, when administered alone, immune modulators need to be dosed frequently, and often at high levels, in order to diffuse to the tumor and exert their effects. The frequent and high dosage may also be avoided by using the combination therapeutic strategies described herein.

Furthermore, the combination strategies described herein are modular and are applicable for a broad range of cancers. For example, the CAR may be engineered to target a wide range of factors (e.g., tumor associated antigens) and the VHH or VHH fusion protein can also be engineered for specific functionalities. Various VHHs can be secreted without need for much additional optimization. Using the combination therapeutic strategies described herein, multiple therapeutic effects can be achieved by administering a single agent (i.e., the engineered cell described herein).

Accordingly, some aspects of the present disclosure provide engineered cells comprising a chimeric antigen receptor (CAR) comprising an extracellular target-binding moiety and an intracellular signaling domain, wherein the engineered cell secrets a VHH or a VHH fusion protein.

An “engineered cell,” as used herein, refers to a non-naturally occurring cell that is engineered (e.g., genetically engineered) to express one or more (e.g., 1, 2, 3, 4, 5, or more) exogenous proteins. The engineered cell of the present disclosure is engineered to express a chimeric antigen receptor (CAR) on its surface. In some embodiments, the engineered cell of the present disclosure expresses more than one (e.g., 2, 3, or more) CARs on its surface. In addition to the chimeric antigen receptor, the engineered cell described herein also expresses and secretes a single domain antibody (e.g., a VH or VHH, including modified variants thereof, such as camelized VHs and humanized VHHs).

In some embodiments, the engineered cell is engineered to express the chimeric antigen receptor and the VHH or VHH fusion protein by delivery into the engineered cell nucleotide sequences encoding the chimeric antigen receptor and the VHH or VHH fusion protein. Any methods of delivering nucleic acids into a cell known in the art may be used, e.g., transformation, transfection, transduction, or electroporation.

In some embodiments, the engineered cell of the present disclosure comprises: (i) a nucleotide sequence encoding the chimeric antigen receptor; and (ii) a nucleotide sequence encoding the VHH or VHH fusion protein. In some embodiments, the nucleotide sequence of (i) is operably linked at the 5′ end to a nucleotide sequence encoding a signal sequence. In some embodiments, the nucleotide sequence of (ii) is operably linked at the 5′ end to a nucleotide sequence encoding a signal sequence. In some embodiments, the nucleotide sequence of (i) is operably linked at the 5′ end to a nucleotide sequence encoding a signal sequence, and the nucleotide sequence of (ii) is operably linked at the 5′ end to a nucleotide sequence encoding a signal sequence. When two coding sequences are “operably linked,” the open reading frames a linked “in frame” such that a fusion protein is produced upon translation of the coding sequences.

A “signal sequence” typically comprises the N-terminal 15-60 amino acids of proteins, and are typically needed for the translocation across the membrane on the secretory pathway and thus universally control the entry of most proteins both in eukaryotes and prokaryotes to the secretory pathway. Signal sequences generally include three regions: an N-terminal region of differing length, which usually comprises positively charged amino acids, a hydrophobic region, and a short carboxy-terminal peptide region. In eukaryotes, the signal sequence of a nascent precursor protein (pre-protein) directs the ribosome to the rough endoplasmic reticulum (ER) membrane and initiates the transport of the growing peptide chain across it. The signal sequence is not responsible for the final destination of the mature protein, however. Secretory proteins devoid of further address tags in their sequence are by default secreted to the external environment. Signal sequences are cleaved from precursor proteins by an endoplasmic reticulum (ER)-resident signal peptidase or they remain uncleaved and function as a membrane anchor. During recent years, a more advanced view of signal sequences has evolved, showing that the functions and immunodorminance of certain signal sequences are much more versatile than previously anticipated.

A signal sequence may have a length of 15-60 amino acids. For example, a signal sequence may have a length of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids. In some embodiments, a signal sequence may have a length of 20-60, 25-60, 30-60, 35-60, 40-60, 45-60, 50-60, 55-60, 15-55, 20-55, 25-55, 30-55, 35-55, 40-55, 45-55, 50-55, 15-50, 20-50, 25-50, 30-50, 35-50, 40-50, 45-50, 15-45, 20-45, 25-45, 30-45, 35-45, 40-45, 15-40, 20-40, 25-40, 30-40, 35-40, 15-35, 20-35, 25-35, 30-35, 15-30, 20-30, 25-30, 15-25, 20-25, or 15-20 amino acids.

Signal sequences that may be used in accordance with the present disclosure are available in the art, e.g., can be found in databases such as signal peptide. In some embodiments, the signal sequence used in accordance with the present disclosure is a CD8 leader sequence. The chimeric antigen receptor comprises a signal sequence for the secretion of its extracellular targeting binding moiety, and the secreted VHH or VHH fusion protein comprises a signal sequence for its secretion.

In some embodiments, the nucleotide sequences of (i) and (ii) are linked, e.g., via a nucleotide sequence that serves as a linker. In some embodiments, when the nucleotide sequence of (i) and (ii) are linked, they are under the control of one promoter. For example, in some embodiments, the nucleotide sequence of (i) is upstream of the nucleotide sequence of (ii), and the nucleotide sequence of (i) is operably linked to a promoter. As such, the nucleotide sequence of (i) and the nucleotide sequence of (ii) are transcribed as one polycistronic mRNA. In these instances, the nucleotide sequence of (i) and the nucleotide sequence of (ii) are linked via a nucleotide sequence encoding a self-cleaving peptide or via an internal ribosome entry site (IRES).

In some embodiments, the nucleotide sequences of (i) and (ii) are linked via a nucleotide sequence encoding a self-cleaving peptide. A “self-cleaving peptide,” as used herein, refers to a peptide that can induce the cleaving of itself from a recombinant protein it is fused to. In some embodiments, the self-cleaving peptide is derived from the 2A region in the genome of a virus (e.g., an Aphthovirus). In some embodiments, the self-cleaving peptide is 18-22 (e.g., 18-22, 18-21, 18-20, 19-22, 19-21, or 20-22) amino acids in length. In some embodiments, the self-cleaving peptide is 18, 19, 20, 21, or 22 amino acids in length. Non-limiting examples of self-cleaving peptide that may be used in accordance with the present disclosure include: P2A (ATNFSLLKQAGDVEENPGP), T2A (EGRGSLLTCGDVEENPGP), E2A (QCTNYALLKLAGDVESNPGP), and F2A (VKQTLNFDLLKLAGDVESNPGP).

Typically, the cleavage is trigged by breaking of peptide bond between the Proline (P) and Glycine (G) in C-terminal of a self-cleaving peptide.

The nucleotide sequences of (i) and (ii) are linked via a nucleotide sequence encoding a self-cleaving peptide such that the chimeric antigen receptor and the VHH or VHH fusion protein are translated as a fusion protein fused via the self-cleaving peptide. The self-cleaving peptide then undergoes self-cleavage, producing a separate chimeric antigen receptor and a VHH or VHH fusion protein. The signal sequence on the chimeric antigen receptor then mediates the secretion of the extracellular targeting moiety of the chimeric receptor, and the signal sequence on the VHH or VHH fusion protein mediates the secretion of the VHH or VHH fusion protein.

In some embodiments, the nucleotide sequences of (i) and (ii) are linked via a nucleotide sequence encoding an internal ribosome entry site (IRES). When the nucleotide sequences of (i) and (ii) are linked via an IRES, the chimeric antigen receptor and the VHH or VHH fusion protein are translated separately. An “internal ribosome entry site (IRES) is a RNA element that allows for translation initiation in a cap-independent manner, as part of the greater process of protein synthesis. In eukaryotic translation, initiation typically occurs at the 5′ end of mRNA molecules, since 5′ cap recognition is required for the assembly of the initiation complex. The presence of an IRES elements allows translation to initiate independent of a 5′ cap. As such, the presence of the IRES in the 3′ fragment of the initial RNA transcript allows expression of the RNA repressor. IRESs are commonly located in the 5′UTR of RNA viruses. Any of these IRES sequences may be used in accordance with the present disclosure. Information regarding the identify and sequences of IRES is available in the art, e.g., in public data bases such as iresite.org. In some embodiments, the IRES is derived from Encephalomyocarditis virus.

Encephalomyocarditis virus IRES Sequence
CCCCTCTCCCTCCCCCCCCCCTAACGTTACTGGCCGAAGCCGCTTGGAAT
AAGGCCGGTGTGCGTTTGTCTATATGTTATTTTCCACCATATTGCCGTCT
TTTGGCAATGTGAGGGCCCGGAAACCTGGCCCTGTCTTCTTGACGAGCAT
TCCTAGGGGTCTTTCCCCTCTCGCCAAAGGAATGCAAGGTCTGTTGAATG
TCGTGAAGGAAGCAGTTCCTCTGGAAGCTTCTTGAAGACAAACAACGTCT
GTAGCGACCCTTTGCAGGCAGCGGAACCCCCCACCTGGCGACAGGTGCCT
CTGCGGCCAAAAGCCACGTGTATAAGATACACCTGCAAAGGCGGCACAAC
CCCAGTGCCACGTTGTGAGTTGGATAGTTGTGGAAAGAGTCAAATGGCTC
ACCTCAAGCGTATTCAACAAGGGGCTGAAGGATGCCCAGAAGGTACCCCA
TTGTATGGGATCTGATCTGGGGCCTCGGTGCACATGCTTTACATGTGTTT
AGTCGAGGTTAAAAAACGTCTAGGCCCCCCGAACCACGGGGACGTGGTTT
TCCTTTGAAAAACACGATGATAA

In some embodiments, the nucleotide sequence of (i) is operably linked to a first promoter and the nucleotide sequence of (ii) is operably linked to a second promoter. As such, the chimeric antigen receptor and the VHH or VHH fusion protein are transcribed and translated separately.

A “promoter” refers to a control region of a nucleic acid sequence at which initiation and rate of transcription of the remainder of a nucleic acid sequence are controlled. A promoter drives expression or drives transcription of the nucleic acid sequence that it regulates. A promoter may also contain sub-regions at which regulatory proteins and molecules may bind, such as RNA polymerase and other transcription factors. Promoters may be constitutive, inducible, activatable, repressible, tissue-specific or any combination thereof. A promoter is considered to be “operably linked” when it is in a correct functional location and orientation in relation to a nucleic acid sequence it regulates to control (“drive”) transcriptional initiation and/or expression of that sequence.

A promoter may be one naturally associated with a gene or sequence, as may be obtained by isolating the 5′ non-coding sequences located upstream of the coding segment of a given gene or sequence. Such a promoter can be referred to as “endogenous.”

In some embodiments, a coding nucleic acid sequence may be positioned under the control of a recombinant or heterologous promoter, which refers to a promoter that is not normally associated with the encoded sequence in its natural environment. Such promoters may include promoters of other genes; promoters isolated from any other cell; and synthetic promoters or enhancers that are not “naturally occurring” such as, for example, those that contain different elements of different transcriptional regulatory regions and/or mutations that alter expression through methods of genetic engineering that are known in the art. In addition to producing nucleic acid sequences of promoters and enhancers synthetically, sequences may be produced using recombinant cloning and/or nucleic acid amplification technology, including polymerase chain reaction (PCR) (see U.S. Pat. Nos. 4,683,202 and 5,928,906).

The promoters that are linked to the nucleotide sequence of (i) and/or (ii) may be constitutive or inducible. An “inducible promoter” refers to a promoter that is characterized by regulating (e.g., initiating or activating) transcriptional activity when in the presence of, influenced by or contacted by an inducer signal. An inducer signal may be endogenous or a normally exogenous condition (e.g., light), compound (e.g., chemical or non-chemical compound) or protein that contacts an inducible promoter in such a way as to be active in regulating transcriptional activity from the inducible promoter. Thus, a “signal that regulates transcription” of a nucleic acid refers to an inducer signal that acts on an inducible promoter. A signal that regulates transcription may activate or inactivate transcription, depending on the regulatory system used. Activation of transcription may involve directly acting on a promoter to drive transcription or indirectly acting on a promoter by inactivation a repressor that is preventing the promoter from driving transcription. Conversely, deactivation of transcription may involve directly acting on a promoter to prevent transcription or indirectly acting on a promoter by activating a repressor that then acts on the promoter. In some embodiments, using inducible promoters in the genetic circuits of the cell state classifier results in the conditional expression or a “delayed” expression of a gene product.

The administration or removal of an inducer signal results in a switch between activation and inactivation of the transcription of the operably linked nucleic acid sequence. Thus, the active state of a promoter operably linked to a nucleic acid sequence refers to the state when the promoter is actively regulating transcription of the nucleic acid sequence (i.e., the linked nucleic acid sequence is expressed). Conversely, the inactive state of a promoter operably linked to a nucleic acid sequence refers to the state when the promoter is not actively regulating transcription of the nucleic acid sequence (i.e., the linked nucleic acid sequence is not expressed).

An inducible promoter of the present disclosure may be induced by (or repressed by) one or more physiological condition(s), such as changes in light, pH, temperature, radiation, osmotic pressure, saline gradients, cell surface binding, and the concentration of one or more extrinsic or intrinsic inducing agent(s). An extrinsic inducer signal or inducing agent may comprise, without limitation, amino acids and amino acid analogs, saccharides and polysaccharides, nucleic acids, protein transcriptional activators and repressors, cytokines, toxins, petroleum-based compounds, metal containing compounds, salts, ions, enzyme substrate analogs, hormones or combinations thereof.

Inducible promoters of the present disclosure include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

Examples of inducible promoters include, without limitation, bacteriophage promoters (e.g. Pls1con, T3, T7, SP6, PL) and bacterial promoters (e.g., Pbad, PmgrB, Ptrc2, Plac/ara, Ptac, Pm), or hybrids thereof (e.g. PLlacO, PLtetO). Examples of bacterial promoters for use in accordance with the present disclosure include, without limitation, positively regulated E. coli promoters such as positively regulated σ70 promoters (e.g., inducible pBad/araC promoter, Lux cassette right promoter, modified lamdba Prm promote, plac Or2-62 (positive), pBad/AraC with extra REN sites, pBad, P(Las) TetO, P(Las) CIO, P(Rhl), Pu, FecA, pRE, cadC, hns, pLas, pLux), σS promoters (e.g., Pdps), σ32 promoters (e.g., heat shock) and σ54 promoters (e.g., glnAp2); negatively regulated E. coli promoters such as negatively regulated σ70 promoters (e.g., Promoter (PRM+), modified lamdba Prm promoter, TetR-TetR-4C P(Las) TetO, P(Las) CIO, P(Lac) IQ, RecA DlexO DLacO1, dapAp, FecA, Pspac-hy, pcI, plux-cI, plux-lac, CinR, CinL, glucose controlled, modified Pr, modified Prm+, FecA, Pcya, rec A (SOS), Rec A (SOS), EmrR_regulated, BetI_regulated, pLac_lux, pTet_Lac, pLac/Mnt, pTet/Mnt, LsrA/cI, pLux/cI, LacI, LacIQ, pLacIQ1, pLas/cI, pLas/Lux, pLux/Las, pRecA with LexA binding site, reverse BBa_R0011, pLacI/ara-1, pLacIq, rrnB P1, cadC, hns, PfhuA, pBad/araC, nhaA, OmpF, RcnR), GS promoters (e.g., Lutz-Bujard LacO with alternative sigma factor σ38), σ32 promoters (e.g., Lutz-Bujard LacO with alternative sigma factor σ32), and σ54 promoters (e.g., glnAp2); negatively regulated B. subtilis promoters such as repressible B. subtilis σA promoters (e.g., Gram-positive IPTG-inducible, Xyl, hyper-spank) and GB promoters. Other inducible microbial promoters may be used in accordance with the present disclosure.

In some embodiments, the nucleotide sequence (i) and the nucleotide sequence of (ii) are on the same vector. A “vector” refers to a nucleic acid (e.g., DNA) used as a vehicle to artificially carry genetic material (e.g., an engineered nucleic acid) into a cell where, for example, it can be replicated and/or expressed. In some embodiments, a vector is an episomal vector (see, e.g., Van Craenenbroeck K. et al. Eur. J. Biochem. 267, 5665, 2000, incorporated by reference herein). A non-limiting example of a vector is a plasmid. Plasmids are double-stranded generally circular DNA sequences that are capable of automatically replicating in a host cell. Plasmid vectors typically contain an origin of replication that allows for semi-independent replication of the plasmid in the host and also the transgene insert. Plasmids may have more features, including, for example, a “multiple cloning site,” which includes nucleotide overhangs for insertion of a nucleic acid insert, and multiple restriction enzyme consensus sites to either side of the insert. Another non-limiting example of a vector is a viral vector (e.g., retroviral, adenoviral, adeno-association, helper-dependent adenoviral systems, hybrid adenoviral systems, herpes simplex, pox virus, lentivirus, Epstein-Barr virus). In some embodiments, the viral vector is derived from an adeno-associated virus (AAV). In some embodiments, the viral vector is derived from an herpes simplex virus (HSV).

In some embodiments, the vector is a retroviral vector. A “retroviral vector” refers to a viral vector derived from the genome of a retrovirus. A retroviral vector contains proviral sequences that can accommodate the gene of interest, to allow incorporation of both into the target cells. The vector also contains viral and cellular gene promoters, such as the CMV promoter, to enhance expression of the gene of interest in the target cells.

In some embodiments, the vector is a lentiviral vector. A “lentiviral vector” is a type of retrovirus that can infect both dividing and nondividing cells because their preintegration complex (virus “shell”) can get through the intact membrane of the nucleus of the target cell. Lentiviruses can be used to provide highly effective gene therapy as lentiviruses can change the expression of their target cell's gene for up to six months. They can be used for nondividing or terminally differentiated cells such as neurons, macrophages, hematopoietic stem cells, retinal photoreceptors, and muscle and liver cells, cell types for which previous gene therapy methods could not be used.

A “chimeric antigen receptor (CAR),” as used herein, refers to an engineered receptor that grafts an selected specificity onto an engineered cell (e.g., an engineered immune cell). The term “chimeric” means that the receptor is composed of parts from different sources. The chimeric antigen receptor of the present disclosure comprises an intracellular signaling domain and an extracellular target-binding moiety.

“An intracellular signaling domain” of a chimeric antigen receptor, as used herein, refers to a domain that, upon activation, stimulates a signaling pathway (transduces a signal) that activates and induces proliferation of an engineered immune cell (e.g., a T cell). In some embodiments, the chimeric antigen receptor further comprises a second (co-stimulatory) intracellular signaling domain that enhances signaling through the signaling pathway created by the first intracellular signaling domain. In some embodiments, the intracellular signaling domain is CD3-zeta. In some embodiments, in chimeric antigen receptors comprising a first and a second intracellular signaling domain (comprising two so-stimulatory domains), one of the intracellular signaling domains is CD3-zeta, and the other of the intracellular signaling domains is selected from CD28, OX40 (CD134), 4-1BB (CD137), and ICOS. An intracellular signaling domain and an intracellular co-signaling domain (which may be referred to collectively as two intracellular co-signaling domains) function together to fully activate an immune cell (each transduce a signal into the immune cell, both which are required to fully activate the immune cell) (see, e.g., June C D et al. Mol. Cell. Biol. 1987; 7:4472-4481). Herein, the terms “intracellular signaling domain” and “intracellular co-signaling domain” may be used interchangeably. For the purpose of the present disclosure, a chimeric antigen receptor is described as having an intracellular signaling domain, if it has either or both of an intracellular signaling domain and an intracellular co-signaling domain.

An “extracellular target-binding moiety” of a chimeric antigen receptor, as used herein, refers to the extracellular domain of the chimeric antigen receptor which has binding specificity to a target molecule (e.g., a tumor specific antigen on a cancer cell). The extracellular target-binding moiety grafts targeting specificity to the chimeric antigen receptor and to the engineered cell expressing the chimeric antigen receptor.

The extracellular target-binding moiety described herein can take various forms. For example, the extracellular target-binding moiety can be an antibody, a single-chain variable fragment (scFv), an antigen binding fragment (Fab), a single domain antibody (e.g., a VH or VHH, including modified variants thereof, such as camelized VHs and humanized VHHs), a diabody, or a synthetic epitope having the broad antibody binding activities described herein.

An “antibody” or “immunoglobulin (Ig)” is a large, Y-shaped protein produced mainly by plasma cells that is used by the immune system to neutralize an exogenous substance (e.g., a pathogens such as bacteria and viruses). Antibodies are classified as IgA, IgD, IgE, IgG, and IgM. “Antibodies” and “antibody fragments” include whole antibodies and any antigen binding fragment (i.e., “antigen-binding portion”) or single chain thereof. In some embodiments, an antibody is a glycoprotein comprising two or more heavy (H) chains and two or more light (L) chains inter-connected by disulfide bonds, or an antigen binding portion thereof. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. An antibody may be a polyclonal antibody or a monoclonal antibody.

The basic 4-chain antibody unit is a heterotetrameric glycoprotein composed of two identical L chains and two H chains (an IgM antibody consists of 5 of the basic heterotetramer unit along with an additional polypeptide called J chain, and therefore contain 10 antigen binding sites, while secreted IgA antibodies can polymerize to form polyvalent assemblages comprising 2-5 of the basic 4-chain units along with J chain). In the case of IgGs, the 4-chain unit is generally about 150,000 daltons. Each L chain is linked to a H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for μ and ε isotypes. Each L chain has at the N-terminus, a variable domain (VL) followed by a constant domain (CL) at its other end. The VL is aligned with the VH and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, (e.g., Basic and Clinical Immunology, 8th edition, Daniel P. Stites, Abba I. Ten and Tristram G. Parslow (eds.), Appleton & Lange, Norwalk, Conn., 1994, page 71 and Chapter 6, incorporated herein by reference).

The L chain from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated α, δ, ε, γ and μ, respectively. The γ and α classes are further divided into subclasses on the basis of relatively minor differences in CH sequence and function, e.g., humans express the following subclasses: IgG1, IgG2, IgG3, IgG4, IgA1, and IgA2.

The V domain mediates antigen binding and define specificity of a particular antibody for its particular antigen. However, the variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a (3-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest, 5th Ed. Public Health Service, National Institutes of Health, Bethesda, Md. (1991), incorporated herein by reference). The constant domains are not involved directly in binding an antibody to an antigen, but exhibit various effector functions, such as participation of the antibody in antibody dependent cellular cytotoxicity (ADCC).

In some embodiments, the extracellular target-binding moiety described herein is a monoclonal antibody. A “monoclonal antibody” is an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present invention may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567). Monoclonal antibodies may also be isolated from phage antibody libraries, e.g., using the techniques described in Clackson et al., Nature, 352:624-628 (1991) and Marks et al., J. Mol. Biol., 222:581-597 (1991), incorporated herein by reference.

The monoclonal antibodies described herein encompass “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, Ape etc.), and human constant region sequences.

In some embodiments, the antibodies are “humanized” for use in human (e.g., as therapeutics). “Humanized” forms of non-human (e.g., rodent) antibodies are chimeric antibodies that contain minimal sequence derived from the non-human antibody. Humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

In some embodiments, the extracellular target-binding moiety described herein comprises an antibody fragment containing the antigen-binding portion of an antibody. The antigen-binding portion of an antibody refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (e.g., as described in Ward et al., (1989) Nature 341:544-546, incorporated herein by reference), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883, incorporated herein by reference). Such single chain antibodies are also intended to be encompassed within the term “antigen-binding portion” of an antibody. These antibody fragments are obtained using conventional techniques known to those with skill in the art, and the fragments are screened for utility in the same manner as are full-length antibodies.

In some embodiments, the extracellular target-binding moiety described herein is a Fc fragment, a Fv fragment, or a single-change Fv fragment. The Fc fragment comprises the carboxy-terminal portions of both H chains held together by disulfides. The effector functions of antibodies are determined by sequences in the Fc region, which region is also the part recognized by Fc receptors (FcR) found on certain types of cells.

The Fv fragment is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

An “antigen binding fragment (Fab)” is the region on an antibody that binds antigens. The Fab is composed of one constant and one variable domain from each of the heavy and light chain polypeptides of the antibody. The antigen binding site is formed by the variable domains of the heavy and light chain antibodies.

A single-chain variable fragment (scFv) is a fusion protein of the variable regions of the heavy (VH) and light chains (VL) of immunoglobulins, connected with a short peptide linker comprising 10-25 amino acids. The linker peptide is usually rich in glycine for flexibility, as well as serine or threonine for solubility, and connects the N-terminus of the VH chain with the C-terminus of the VL chain, or vice versa. The scFv retains the specificity of the original immunoglobulin, despite the addition of the linker and removal of the constant regions. In some embodiments, the sFv polypeptide further comprises a polypeptide linker between the VH and VL domains which enables the sFv to form the desired structure for antigen binding (e.g., as described in Pluckthun in The Pharmacology of Monoclonal Antibodies, vol. 113, Rosenburg and Moore eds., Springer-Verlag, New York, pp. 269-315 (1994); Borrebaeck 1995, incorporated herein by reference).

A single domain antibody is an antibody fragment consisting of a monomeric VH or VL domain which retains selective binding to a specific antigen. Single domain antibodies are small (˜12-15 kilodaltons), readily cross the blood-brain barrier, have improved solubility, and are thermostable relative to full-length antibodies.

A diabody is a dimeric antibody fragment designed to form two antigen binding sites. Diabodies are composed of two single-chain variable fragments (scFvs) in the same polypeptide connected by a linker peptide which is too short (˜3-6 amino acids) to allow pairing between the two domains on the same chain, forcing the domains to pair with complementary domains of another chain to form two antigen binding sites. Alternately, the two scFvs can also be connected with longer linkers, such as leucine zippers.

In some embodiments, the extracellular target-binding moiety described herein is single chain antibody (e.g., a heavy chain-only antibody). It is known that Camilids produce heavy chain-only antibodies (e.g., as described in Hamers-Casterman et al., 1992, incorporated herein by reference). The single-domain variable fragments of these heavy chain-only antibodies are termed VHHs or nanobodies. VHHs retain the immunoglobulin fold shared by antibodies, using three hypervariable loops, CDR1, CDR2 and CDR3, to bind to their targets. Many VHHs bind to their targets with affinities similar to conventional full-size antibodies, but possess other properties superior to them. Therefore, VHHs are attractive tools for use in biological research and therapeutics. VHHs are usually between 10 to 15 kDa in size, and can be recombinantly expressed in high yields, both in the cytosol and in the periplasm in E. coli. VHHs can bind to their targets in mammalian cytosol. A VHH fragment (e.g., NANOBODY®) is a recombinant, antigen-specific, single-domain, variable fragment derived from camelid heavy chain antibodies. Although they are small, VHH fragments retain the full antigen-binding capacity of the full antibody. VHHs are small in size, highly soluble and stable, and have greater set of accessible epitopes, compared to traditional antibodies. They are also easy to use as the extracellular target-binding moiety of the chimeric receptor described herein, because no reformatting is required.

The extracellular target-binding moiety of the chimeric antigen receptor can be engineered to target any antigens present in a target cell (e.g., on the surface of a target cell). In some embodiments, the extracellular target-binding moiety of the chimeric antigen receptor binds a tumor-associated antigen. In some embodiments, for tumors that have few known tumor-associated antigens (e.g., solid tumor), the extracellular target-binding moiety of the chimeric antigen receptor described herein target the tumor microenvironment (e.g., tumor neovasculature and stroma).

A “tumor-associated antigen” refers to an antigenic substance produced by a cancer cell and triggers an immune response in the host. In some embodiments, the cancer antigen is a protein that specifically expresses or is upregulated in a cancer cell, as compared to a non-cancerous cell. Exemplary cancer antigens include, without limitation: MAGE family members, NY-ESO-1, tyrosinase, Melan-A/MART-1, prostate cancer antigen, Her-2/neu, Survivin, Telomerase, WT1, CEA, gp100, Pmel17, mammaglobin-A, NY-BR-1, ERBB2, OA1, PAP, RAB38/NY-MEL-1, TRP-1/gp75, TRP-2, CD33, BAGE-1, D393-CD20n, cyclin-A1, GAGE-1, GAGE-2, GAGE-8, GnTVf, HERV-K-MEL, KK-LC-1, KM-HN-1, LAGE-1, LY6K, MAGE-A1, MAGE-A2, MAGE-A3, MAGE-A4, MAGE-A6, MAGE-A9, MAGE-A10, MAGE-A12, MAGE-C1, MAGE-C2, mucink, NA88-A, SAGE, sp17, SSX-2, SSX-4, surviving, TAG-1, TAG-3, TRAG-3, XAGE-1b, BCR-ABl, adipophiln, AIM-2, ALDH1A1, BCLX(L), BING-4, CALCA, CD45, CD274, CPSF, cyclin D1, DKK1, ENAH, EpCAM, EphA3, EZH2, FGF5, glypican-3, G250, HER-2, HLA-DOB, hepsin, IDO1, IGF2B3, IL12Ralpha2, intestinal carboyxyl esterase, alpha-foetoprotein, kallikrein 4, KIF20A, Lengsin, M-CSF, M-CSP, mdm-2, Meloe, midkine, MMP-2, MMP-7, MUC1, MUC5AC, p53, PAX5, PBF, PRAME, PSMA, RAGE-1, RGS5, RhoC, RNF43, RU2AS, secerinel, SOX10, STEAP1, telomerase, TPBG, mesothelin, Axl, and VEGF.

In some embodiments, the tumor associated antigen targeted by the extracellular target-binding moiety of the chimeric antigen receptor is selected from the group consisting of: PDL1, EIIIB fibronectin, CEA, PSMA, AXL, HER2, CD133, Muc1, Muc16, Siglec15, and mesothelin.

In some embodiments, the extracellular target-binding moiety of the chimeric antigen receptor binds Programmed death-ligand 1 (PD-L1). PD-L1 has been shown to be highly upregulated in several solid tumors (e.g., melanoma, renal cell carcinoma (RCC), non-small cell lung cancer, thymona, ovarian cancer, or colorectal cancer (e.g., as described in Partel et al., Molecular Cancer Therapeutics, Volume 14, Issue 4, 2015, incorporated herein by reference). In some embodiments, the extracellular target-binding moiety of the chimeric antigen receptor is a VHH that binds PD-L1 (e.g., the B3 or A12 VHHs as described in Ingram et al., Nat Commun. 2017; 8: 647, incorporated herein by reference).

In some embodiments, the extracellular target-binding moiety of the chimeric antigen receptor binds EIIIB fibronectin. A splice variant of EIIIB fibronectin present in neovasculature and tumor stroma and has been shown to be produced by endothelial cells in cancer (e.g., as described in Bordeleau et al., PNAS, Vol. 112, No. 7, 8314-8319, 2015 incorporated herein by reference). EIIIB fibronectin is highly conserved in all vertebrates. In some embodiments, the extracellular target-binding moiety of the chimeric antigen receptor is a VHH that binds EIIIB fibronectin.

In some embodiments, the extracellular target-binding moiety of the chimeric antigen receptor is a bi-specific antibody (i.e., an antibody that binds two antigens). In some embodiments, the extracellular target-binding moiety of the chimeric antigen receptor is a bi-specific antibody (e.g., a bi-specific VHH) that binds both PD-L1 and EIIIB fibronectin.

In some embodiments, the extracellular target-binding moiety of the chimeric antigen receptor targets binds an autoimmune antigen. An “autoimmune antigen” refers to an antigen that is derived from one's own body (a self-antigen). In some embodiments, the autoimmune antigen is selected from the group consisting of: antigen-specific T-cell receptors, B cell receptors, and insulin receptor.

An “antigen-specific T-cell receptor” or “T-cell receptor (TCR)” refers to is a cell-surface receptor on T cells and is responsible for recognizing fragments of antigen as peptides bound to major histocompatibility complex (MHC) molecules. The binding between TCR and antigen peptides is of relatively low affinity and is degenerate. Many TCRs recognize the same antigen peptide and many antigen peptides are recognized by the same TCR. Genes encoding TCRs can be recombined to produce TCRs specific for a certain antigen.

A “B cell receptor” refers to immunoglobulin molecules that form a type 1 transmembrane receptor protein usually located on the outer surface of a lymphocyte type known as B cells. Through biochemical signaling and by physically acquiring antigens from the immune synapses, the BCR controls the activation of B-cell.

An “insulin receptor” refers to a transmembrane receptor that is activated by insulin, IGF-I, IGF-II and belongs to the large class of tyrosine kinase receptors.

The engineered cell described herein comprises a chimeric antigen receptor and secretes a VHH or VHH fusion protein. In some embodiments, the secreted VHH or VHH fusion protein is designed to improve the efficacy of the chimeric antigen receptor. The chimeric antigen receptor in the engineered cell targets the cell to the target site (e.g., a tumor cell), where the supporting VHH or VHH fusions are secreted, further enhancing the therapeutic potency of the engineered cell.

In some embodiments, the secreted VHH or VHH fusion protein binds an immune checkpoint protein. An “immune checkpoint protein” is a protein in the immune system that either enhances an immune response signal (co-stimulatory molecules) or reduces an immune response signal. Many cancers protect themselves from the immune system by exploiting the inhibitory immune checkpoint proteins to inhibit the T cell signal. Exemplary inhibitory checkpoint proteins include, without limitation, Cytotoxic T-Lymphocyte-Associated protein 4 (CTLA-4), Programmed Death 1 receptor (PD-1), T-cell Immunoglobulin domain and Mucin domain 3 (TIM3), Lymphocyte Activation Gene-3 (LAG3), V-set domain-containing T-cell activation inhibitor 1 (VTVN1 or B7-H4), Cluster of Differentiation 276 (CD276 or B7-H3), B and T Lymphocyte Attenuator (BTLA), Galectin-9 (GALS), Checkpoint kinase 1 (Chk1), Adenosine A2A receptor (A2aR), Indoleamine 2,3-dioxygenase (IDO), Killer-cell Immunoglobulin-like Receptor (KIR), Lymphocyte Activation Gene-3 (LAG3), and V-domain Ig suppressor of T cell activation (VISTA).

Some of these immune checkpoint proteins need their cognate binding partners, or ligands, for their immune inhibitory activity. For example, A2AR is the receptor of adenosine A2A and binding of A2A to A2AR activates a negative immune feedback loop. As another example, PD-1 associates with its two ligands, PD-L1 and PD-L2, to down regulate the immune system by preventing the activation of T-cells. PD-1 promotes the programmed cell death of antigen specific T-cells in lymph nodes and simultaneously reduces programmed cell death of suppressor T cells, thus achieving its immune inhibitory function. As yet another example, CTLA4 is present on the surface of T cells, and when bound to its binding partner CD80 or CD86 on the surface of antigen-present cells (APCs), it transmits an inhibitory signal to T cells, thereby reducing the immune response. For the purpose of the present disclosure, these cognate binding partners are also immune checkpoint proteins and can be targeted by the secreted VHH or VHH fusion protein. In some embodiments, the VHH or VHH fusion protein binds an immune checkpoint protein selected from CTLA4, PD1, PDL1, TIM3, LAG3, VISTA, and CD47.

In some embodiments, the secreted VHH or VHH fusion protein binds Cluster of differentiation 47 (CD47). CD47 is a ubiquitously expressed glycoprotein of the immunoglobulin superfamily that plays a critical role in self-recognition. Various solid and hematologic cancers exploit CD47 expression in order to evade immunological eradication, and its overexpression is clinically correlated with poor prognoses. One essential mechanism behind CD47-mediated immune evasion is that it can trigger an anti-phagocytic signal, allowing tumor cells to evade phacytosis by macrophages. By targeting CD47 on the surface of the cancer cell, innate immunity against the cancer cell is improved through macrophage engagement.

In some embodiments, the secreted VHH or VHH fusion protein binds a tumor-associated antigen. Any of the tumor-associated antigens described herein may be targeted by the secreted VHH or VHH fusion protein. In some embodiments, the secreted VHH or VHH associate protein binds EIIIB fibronectin, Siglec15, VEGF(R), HER2, PSMA, AXL, Muc1, or Muc16.

In some embodiments, the secreted VHH or VHH fusion protein binds an immune cell associated antigen. Non-limiting examples of immune cell associated antigens include: MHCI/II, CD40L, CD40, and CD80/CD86.

In some embodiments, the engineered cell secretes a VHH (e.g., the engineered cell comprises a nucleotide sequence encoding a VHH). In some embodiments, the engineered cell secretes a VHH fusion protein (e.g., the engineered cell comprises a nucleotide sequence encoding a VHH fusion protein). The small size and high solubility of VHHs make them suitable for fusion to other molecules (e.g., therapeutic polypeptides) for secretion by the engineered cell.

In some embodiments, the VHH fusion protein comprises a VHH fused to a fragment crystallizable region (Fc). A “fragment crystallizable region (Fc)” refers to the tail region of an antibody that interacts with cell surface receptors called Fc receptors and some proteins of the complement system, which allows antibodies to activate the immune system. In some embodiments, the Fc domain is a Fc domain from an IgG, IgA, IgM, IgD, or IgE, or variants thereof. In some embodiments, the Fc domain is an Fc portion of human IgG1.

Fc portion of human IgG1
(SEQ ID NO: 21)
THTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCK
VSNKALPVPIEKTISKAKGQPREPQVYTLPPSREEMTKNQVSLTCLVKGF
YPSDIAVEWESNGQPENNYKTTPPVLDSDGPFFLYSKLTVDKSRWQQGNV
FSCSVMHEALHNHYTQKSLSLSPGK

In some embodiments, the Fc domain fused to the VHH in the VHH fusion protein comprises an amino acid sequence that is at least 80%, at least 85%, at least 90%, at least 95%, at least 99% or more identical to the amino acid sequence of SEQ ID NO: 21. In some embodiments, the Fc domain fused to the VHH in the VHH fusion protein comprises an amino acid sequence that is 80%, 85%, 90%, 95%, or 99% identical to the amino acid sequence of SEQ ID NO: 21. In some embodiments, the Fc domain fused to the VHH in the VHH fusion protein comprises the amino acid sequence of SEQ ID NO: 1. In some embodiments, the Fc domain fused to the VHH in the VHH fusion protein consists of the amino acid sequence of SEQ ID NO: 1.

In some embodiments, fusing the VHH to an Fc domain increases the stability of the VHH (e.g., by at least 20%, at least 30%, at least 40%, at least 50%, at least 60%, at least 70%, at least 80%, at least 90%, at least 100%, at least 2-fold, at least 5-fold, at least 10-fold, at least 100-fold, or more), compared to the VHH alone. In some embodiments, fusing the VHH to an Fc domain decreases the cell toxicity of the VHH (e.g., by at least 20%, at least 30%, at least 40%, at least 50%, at least 60%, at least 70%, at least 80%, at least 90%, at least 95%, at least 99%, or more), compared to the VHH alone.

Other proteins or polypeptides that may be fused to the secreted VHH described herein include, without limitation: enzymes, cytokines, and different VHHs.

When a VHH fusion protein is secreted, the binding specificity is determined by the VHH portion of the fusion protein, except when the VHH is fused to another VHH (e.g., a VHH that binds a different target). When the VHH fusion protein comprises two VHHs with different binding specificity fused together, the VHH fusion protein is a bi-specific VHH (e.g., a bi-specific VHH that binds both PD-L1 and EIIIB fibronectin, a bi-specific VHH that binds CD47 and CTLA-4, or a bi-specific VHH that binds both TIM3 and LAGS. In some embodiments, the two VHHs are fused via a cleavable peptide (e.g., the P2A peptide) and the two VHHs can be separated by cleaving the peptide after secretion.

Exemplary VHHs that may be used in accordance with the present disclosure, either as the extracellular target-binding moiety or as the secreted VHH or VHH fusion protein, and their gene and amino acid sequences are provided in Table 1.

TABLE 1
Non-limiting, exemplary VHHs

| | Amino acid | Nucleotide sequence encoding VHH with |
|---|---|---|
| VHH | Sequence | signal sequence |
| Anti-CD47 (A4) | QVQLVESGGGLV | CAAGTCCAGTTGGTGGAGTCTGGTGGTGGCCTTG |
| | EPGGSLRLSCAA | TGGAGCCTGGTGGCAGCCTGCGCCTGAGCTGTGC |
| | SGIIFKINDMGW | CGCCAGCGGGATAATTTTCAAGATCAACGATATG |
| | YRQAPGKRREW | GGTTGGTACAGACAGGCCCCCGGCAAGAGACGG |
| | VAASTGGDEAIY | GAATGGGTAGCCGCTAGTACTGGCGGTGACGAGG |
| | RDSVKDRFTISR | CTATATATCGCGATTCTGTGAAGGATCGGTTCACT |
| | DAKNSVFLQMN | ATCTCCCGCGACGCCAAAAATTCCGTCTTCCTGCA |
| | SLKPEDTAVYYC | GATGAATAGCTTGAAACCTGAGGACACAGCGGTT |
| | TAVISTDRDGTE | TACTACTGTACCGCCGTGATTTCTACCGACAGGG |
| | WRRYWGQGTQV | ACGGCACTGAATGGCGGCGCTACTGGGGCCAAGG |
| | TVSS (SEQ ID NO: | GACGCAGGTCACGGTGTCCAGC (SEQ ID NO: 11) |
| | 1) | |
| Anti-PD-L1 | QVQLVESGGGLV | CAAGTGCAGCTTGTCGAATCCGGCGGCGGCCTCG |
| (A12) | QAGGSLRLSCTA | TGCAGGCTGGAGGCAGCCTCCGATTGAGCTGCAC |
| | SGSTFSRNAMA | TGCTTCAGGGAGTACCTTCTCACGGAATGCAATG |
| | WFRQAPGKEREF | GCCTGGTTCAGGCAGGCCCCTGGCAAGGAACGCG |
| | VSGISRTGTNSY | AATTTGTCTCTGGTATCAGCCGGACGGGTACAAA |
| | DADSVKGRFTIS | CTCCTATGATGCTGATAGTGTAAAGGGTCGGTTC |
| | KDNAKNTVTLQ | ACGATTTCCAAGGACAACGCAAAAAACACTGTGA |
| | MNSLKPEDTAIY | CTCTTCAAATGAACTCACTGAAGCCGGAGGACAC |
| | YCALSQTASVAT | CGCCATATATTATTGTGCCTTGAGTCAGACGGCCA |
| | TERLYPYWGQG | GCGTGGCCACCACAGAGCGACTCTATCCCTACTG |
| | TQVTVSS (SEQ | GGGCCAGGGAACACAGGTGACTGTGTCTAGT |
| | ID NO: 2) | (SEQ ID NO: 12) |
| Anti-EIIB | QVQLVETGGGL | CAGGTGCAGCTCGTGGAGACTGGGGGAGGCTTGG |
| fibronectin (B2) | VQAGGSLRLSCA | TGCAGGCTGGGGGGTCTCTGAGACTCTCCTGTGC |
| | ASGSTFSHNAGG | AGCCTCTGGAAGCACATTCAGTCATAATGCCGGC |
| | WYRQAPEKQRE | GGCTGGTACCGCCAGGCTCCAGAAAAGCAGCGCG |
| | LVAGISSDGNINY | AGTTGGTCGCAGGGATTAGTAGTGATGGTAACAT |
| | ADSVKDRFTISR | CAACTATGCGGACTCCGTGAAGGACCGATTCACC |
| | DNASNTMYLQM | ATCTCCAGAGACAACGCCAGCAACACGATGTATC |
| | NNLKPEDTAVYV | TACAAATGAACAACCTGAAACCTGAGGACACGGC |
| | CNIRGSYGNTYY | CGTCTATGTCTGTAATATCAGGGGATCGTACGGT |
| | SRWGQGTQVTV | AATACCTATTACAGTCGGTGGGGCCAGGGGACCC |
| | SS (SEQ ID NO: 3) | AGGTCACCGTCTCCTCA (SEQ ID NO: 13) |
| Anti-CTLA4 | QVQLQESGGGLA | CAGGTGCAGCTGCAGGAGTCTGGAGGAGGGTTGG |
| (H11) | QPGGSLRLSCAA | CGCAGCCTGGGGGGTCTCTGAGACTCTCCTGTGC |
| | SGSTISSVAVGW | AGCCTCTGGAAGCACGATCAGTAGCGTCGCCGTG |
| | YRQTPGNQREW | GGCTGGTACCGCCAGACTCCAGGGAATCAGCGCG |
| | VATSSTSSTTATY | AGTGGGTCGCCACTAGTAGCACGAGTAGTACTAC |
| | ADSVKGRFTISR | CGCAACGTATGCTGACTCCGTGAAGGGCCGATTC |
| | DNAKNTIYLQM | ACCATCTCCAGAGACAACGCCAAGAACACGATCT |
| | NSLKPEDTAVYY | ATCTGCAAATGAACAGCCTGAAACCTGAGGACAC |
| | CKTGLTNWGQG | GGCCGTCTATTACTGTAAAACAGGCCTGACTAAT |
| | TQVTVSS (SEQ | TGGGGCCAGGGGACCCAGGTCACCGTCTCCTCA |
| | ID NO: 4) | (SEQ ID NO: 14) |
| Anti-PD-L1 | QVQLQESGGGLV | CAGGTGCAGCTGCAGGAGTCGGGGGGAGGCTTGG |
| (B3) | QPGGSLRLSCTA | TGCAGCCTGGGGGGTCTCTGAGACTTTCCTGTACA |
| | SGFTFSMHAMT | GCCTCTGGATTCACCTTCAGTATGCATGCCATGAC |
| | WYRQAPGKQRE | CTGGTACCGCCAGGCTCCAGGGAAGCAGCGCGAG |
| | LVAVITSHGDRA | TTGGTCGCAGTTATTACTAGTCATGGTGATAGGGC |
| | NYTDSVRGRFTIS | AAACTATACAGACTCCGTGAGGGGCCGATTCACC |
| | RDNTKNMVYLQ | ATCTCCAGAGACAATACCAAGAACATGGTGTATC |
| | MNSLKPEDTAVY | TGCAAATGAACAGCCTGAAACCTGAGGACACGGC |
| | YCNVPRYDSWG | CGTGTATTATTGTAATGTCCCCCGGTATGACTCCT |
| | QGTQVTVSS | GGGGCCAGGGGACCCAGGTCACCGTCTCCTCA |
| | (SEQ ID NO: 5) | (SEQ ID NO: 15) |
| Anti-TIM3 | QVQLVESGGGLV | CAGGTGCAGCTCGTGGAGTCGGGGGGAGGCTTGG |
| (mH2) | QAGGSLRLSCAA | TGCAGGCTGGGGGGTCTCTGAGACTCTCCTGTGC |
| | SGFTFDDYAIGW | AGCCTCTGGATTCACTTTCGATGATTATGCCATAG |
| | FRQAPGKEREGV | GCTGGTTCCGCCAGGCCCCAGGGAAGGAGCGTGA |
| | SCISSSDGSTYYT | GGGGGTCTCATGTATTAGTAGTAGTGATGGTAGC |
| | DSVKGRFTISSDN | ACATACTATACAGACTCCGTGAAGGGCCGATTCA |
| | AKNTVYLQMNS | CCATCTCCAGTGACAACGCCAAGAACACGGTGTA |
| | LKPEDTAVYYCA | TCTGCAAATGAACAGCCTGAAACCTGAGGACACG |
| | ADTTFFGCSLNR | GCCGTTTATTACTGTGCAGCGGACACCACTTTCTT |
| | DYDYWGQGTQV | CGGCTGCTCTCTGAACCGGGACTATGACTACTGG |
| | TVSS (SEQ ID NO: | GGCCAGGGGACCCAGGTCACCGTCTCCTCA (SEQ |
| | 6) | ID NO: 16) |
| Anti-human | QVQLVESGGGM | CAGGTGCAGCTCGTGGAGTCGGGTGGAGGTATGG |
| TIM3 (hH6) | VQPGDSLRLSCV | TGCAACCTGGGGACTCTCTGAGGCTCTCCTGTGTA |
| | ASGRTGSSYIIGW | GCCTCTGGACGCACCGGCAGTAGCTATATCATAG |
| | FRQAPGKEREFV | GCTGGTTCCGCCAGGCTCCAGGAAAGGAGCGTGA |
| | ARVSPSGGTRDY | GTTTGTAGCGCGTGTTTCACCGAGCGGCGGTACC |
| | ADSVKGRFTVSR | AGAGACTATGCAGACTCCGTGAAGGGACGATTCA |
| | DNAKNTVYLQM | CCGTCTCCAGAGACAACGCCAAAAACACGGTGTA |
| | DRLKPEDTAIYT | CCTGCAAATGGACAGGCTGAAACCTGAAGACACG |
| | CAAAGGKWTAD | GCCATTTATACCTGTGCTGCGGCTGGGGGGAAAT |
| | SGEYNYWGQGT | GGACAGCGGATTCGGGAGAGTATAACTACTGGGG |
| | QVTVSS (SEQ ID | CCAGGGGACCCAGGTCACCGTCTCCTCA (SEQ ID |
| | NO: 7) | NO: 17) |
| Anti-MHCII | QVQLQESGGGLV | CAGGTGCAGCTGCAGGAGTCAGGGGGAGGATTG |
| (VHH7) | QAGDSLRLSCAA | GTGCAGGCTGGGGACTCTCTGAGACTCTCCTGCG |
| | SGRTFSRGVMG | CAGCCTCTGGACGCACCTTCAGTCGCGGTGTAAT |
| | WFRRAPGKEREF | GGGCTGGTTCCGCCGGGCTCCAGGGAAGGAGCGT |
| | VAIFSGSSWSGRS | GAGTTTGTAGCAATCTTTAGCGGGAGTAGCTGGA |
| | TYYSDSVKGRFT | GTGGTCGTAGTACATACTATTCAGACTCCGTAAA |
| | ISRDNAKNTVYL | GGGCCGATTCACCATCTCCAGAGACAACGCCAAG |
| | QMNGLKPEDTA | AACACGGTGTATCTGCAAATGAACGGCCTGAAAC |
| | VYYCAAGYPEA | CTGAGGACACGGCCGTTTATTACTGTGCAGCGGG |
| | YSAYGRESTYDY | ATATCCGGAGGCGTATAGCGCCTATGGTCGGGAG |
| | WGQGTQVTVS | AGTACATATGACTACTGGGGCCAGGGGACCCAGG |
| | (SEQ ID NO: 8) | TCACCGTCTC (SEQ ID NO: 18) |
| Anti-GFP (Enh) | QVQLQESGGALV | CAGGTGCAGCTGCAGGAATCGGGTGGTGCCCTGG |
| | QPGGSLRLSCAA | TTCAGCCGGGTGGTAGCCTGCGTCTGTCGTGTGCT |
| | SGFPVNRYSMR | GCGTCGGGTTTTCCGGTTAACCGTTATAGCATGCG |
| | WYRQAPGKERE | TTGGTACCGTCAGGCACCGGGTAAAGAACGTGAA |
| | WVAGMSSAGDR | TGGGTCGCGGGCATGAGCTCTGCCGGTGATCGTA |
| | SSYEDSVKGRFTI | GTTCCTATGAAGACTCAGTGAAAGGTCGCTTTAC |
| | SRDDARNTVYLQ | CATTTCGCGTGATGACGCACGCAACACGGTGTAC |
| | MNSLKPEDTAVY | CTGCAAATGAATAGTCTGAAACCGGAAGATACCG |
| | YCNVNVGFEYW | CTGTTTATTACTGTAATGTTAATGTCGGCTTTGAA |
| | GQGTQVTVSS | TACTGGGGTCAGGGCACGCAGGTCACCGTCTCCT |
| | (SEQ ID NO: 9) | CA (SEQ ID NO: 19) |
| Anti-CDPK1 | QVQLHESGGGLV | CAGGTGCAGCTGCATGAGTCAGGGGGAGGATTGG |
| (1B7) | QPGESLRLSCVA | TGCAGCCTGGGGAGTCTCTGAGACTTTCCTGCGTA |
| | SGFTLDHSAVGW | GCCTCTGGATTCACTCTGGATCATTCTGCCGTCGG |
| | FRQVPGKEREKL | CTGGTTCCGCCAGGTCCCCGGGAAGGAGCGTGAG |
| | LCINANGVSLDY | AAACTCTTGTGCATTAACGCTAACGGTGTTAGCCT |
| | ADSIKGRFTISRD | GGACTATGCAGACTCCATTAAGGGCCGATTCACC |
| | NAKNTVYLQMN | ATCTCTCGGGACAACGCCAAGAACACGGTCTATC |
| | DLKPEDTATYSC | TGCAGATGAACGACCTGAAACCTGAGGACACAGC |
| | AATREFCSAYVF | CACATATAGCTGTGCAGCAACGAGAGAATTCTGT |
| | LYEHWGQGTQV | TCAGCTTATGTGTTCCTATATGAACACTGGGGCCA |
| | TVSS (SEQ ID NO: | GGGGACCCAGGTCACCGTCTCCTCA (SEQ ID NO: |
| | 10) | 20) |

In some embodiments, the VHH used in accordance with the present disclosure, either as the extracellular target-binding moiety or as the secreted VHH, comprises an amino acid sequence that is at least 80%, at least 85%, at least 90%, at least 95%, or at least 99% identical to the amino acid sequence of any one of SEQ ID NOs: 1-10. In some embodiments, the VHH used in accordance with the present disclosure, either as the extracellular target-binding moiety or as the secreted VHH, comprises an amino acid sequence that is 80%, 85%, 90%, 95%, or 99% identical to the amino acid sequence of any one of SEQ ID NOs: 1-10. In some embodiments, the VHH used in accordance with the present disclosure, either as the extracellular target-binding moiety or as the secreted VHH, comprises the amino acid sequence of any one of SEQ ID NOs: 1-10). In some embodiments, the VHH used in accordance with the present disclosure, either as the extracellular target-binding moiety or as the secreted VHH, consists of the amino acid sequence of any one of SEQ ID NOs: 1-10).

In some embodiments, the engineered cell secretes a VHH fusion protein comprising a VHH fused to a Fc, wherein the VHH comprises an amino acid sequence that is at least 80%, at least 85%, at least 90%, at least 95%, or at least 99% identical to the amino acid sequence of any one of SEQ ID NOs: 1-10, and the Fc comprises an amino acid sequence that is at least 80%, at least 85%, at least 90%, at least 95%, or at least 99% identical to SEQ ID NO: 21. In some embodiments, the engineered cell secretes a VHH fusion protein comprising a VHH fused to a Fc, wherein the VHH comprises an amino acid sequence that is 80%, 85%, 90%, 95%, or 99% identical to the amino acid sequence of any one of SEQ ID NOs: 1-10, and the Fc comprises an amino acid sequence that is 80%, 85%, 90%, 95%, or 99% identical to SEQ ID NO: 21. In some embodiments, the engineered cell secretes a VHH fusion protein comprising a VHH fused to a Fc, wherein the VHH comprises the amino acid sequence of any one of SEQ ID NOs: 1-10, and the Fc comprises the amino acid sequence of SEQ ID NO: 21. In some embodiments, the engineered cell secretes a VHH fusion protein comprising a VHH fused to a Fc, wherein the VHH consists of the amino acid sequence of any one of SEQ ID NOs: 1-10, and the Fc consists of the amino acid sequence of SEQ ID NO: 21.

In some embodiments, the engineered cell comprises a chimeric antigen binding receptor comprising an extracellular target-binding moiety that binds PD-L1 and an intracellular signaling domain, and secretes a VHH or VHH fusion protein that binds CD47. In some embodiments, the engineered cell comprises a chimeric antigen binding receptor comprising an extracellular target-binding moiety that is a an anti-PD-L1 VHH (e.g., A12, B3, or variants thereof) and an intracellular signaling domain, and secretes an anti-CD47 VHH (e.g., A4 or variants thereof) or an anti-CD47 VHH fusion protein (e.g., A4-Fc or variants thereof).

In some embodiments, the engineered cell comprises a chimeric antigen binding receptor comprising an extracellular target-binding moiety that binds PD-L1 and an intracellular signaling domain, and secretes a VHH or VHH fusion protein that binds CTLA4. In some embodiments, the engineered cell comprises a chimeric antigen binding receptor comprising an extracellular target-binding moiety that is a an anti-PD-L1 VHH (e.g., A12, B3, or variants thereof) and an intracellular signaling domain, and secretes an anti-CTLA4 VHH (e.g., H11 or variants thereof) or an anti-CTLA4 VHH fusion protein (e.g., Hi i-Fc or variants thereof).

In some embodiments, the engineered cell comprises a chimeric antigen binding receptor comprising an extracellular target-binding moiety that binds PD-L1 and an intracellular signaling domain, and secretes a VHH or VHH fusion protein that binds PD-1. In some embodiments, the engineered cell comprises a chimeric antigen binding receptor comprising an extracellular target-binding moiety that is a an anti-PD-L1 VHH (e.g., A12, B3, or variants thereof) and an intracellular signaling domain, and secretes an anti-PD-1 VHH or an anti-PD-1 VHH fusion protein (e.g., Fc fusion or variants thereof).

In some embodiments, the engineered cell comprises a chimeric antigen binding receptor comprising an extracellular target-binding moiety that binds PD-L1 and an intracellular signaling domain, and secretes a VHH or VHH fusion protein that binds TIM3. In some embodiments, the engineered cell comprises a chimeric antigen binding receptor comprising an extracellular target-binding moiety that is a an anti-PD-L1 VHH (e.g., A12, B3, or variants thereof) and an intracellular signaling domain, and secretes an anti-TIM3 VHH (e.g., mH2, hH6 or variants thereof) or an anti-TIM3 VHH fusion protein (e.g., mH2-Fc, hH6-Fc or variants thereof).

In some embodiments, the engineered cell comprises a chimeric antigen binding receptor comprising an extracellular target-binding moiety that binds PD-L1 and an intracellular signaling domain, and secretes a VHH or VHH fusion protein that binds EIIIB fibronectin. In some embodiments, the engineered cell comprises a chimeric antigen binding receptor comprising an extracellular target-binding moiety that is a an anti-PD-L1 VHH (e.g., A12, B3, or variants thereof) and an intracellular signaling domain, and secretes an anti-EIIIB fibronectin VHH (e.g., B2 or variants thereof) or an anti-EIIIB fibronectin VHH fusion protein (e.g., B2-Fc or variants thereof).

In some embodiments, the engineered cell comprises a chimeric antigen binding receptor comprising an extracellular target-binding moiety that binds EIIIB fibronectin and an intracellular signaling domain, and secretes a VHH or VHH fusion protein that binds CD47. In some embodiments, the engineered cell comprises a chimeric antigen binding receptor comprising an extracellular target-binding moiety that is a an anti-EIIIB fibronectin VHH (e.g., B2 or variants thereof) and an intracellular signaling domain, and secretes an anti-CD47 VHH (e.g., A4 or variants thereof) or an anti-CD47 VHH fusion protein (e.g., A4-Fc or variants thereof).

In some embodiments, the engineered cell comprises a chimeric antigen binding receptor comprising an extracellular target-binding moiety that binds EIIIB fibronectin and an intracellular signaling domain, and secretes a VHH or VHH fusion protein that binds CTLA-4. In some embodiments, the engineered cell comprises a chimeric antigen binding receptor comprising an extracellular target-binding moiety that is a an anti-EIIIB fibronectin VHH (e.g., B2 or variants thereof) and an intracellular signaling domain, and secretes an anti-CTLA-4 VHH (e.g., H11 or variants thereof) or an anti-CTLA4 VHH fusion protein (e.g., Hi i-Fc or variants thereof).

In some embodiments, the engineered cell comprises a chimeric antigen binding receptor comprising an extracellular target-binding moiety that binds EIIIB fibronectin and an intracellular signaling domain, and secretes a VHH or VHH fusion protein that binds PD-1. In some embodiments, the engineered cell comprises a chimeric antigen binding receptor comprising an extracellular target-binding moiety that is a an anti-EIIIB fibronectin VHH (e.g., B2 or variants thereof) and an intracellular signaling domain, and secretes an anti-PD-1 VHH or an anti-PD-1 VHH fusion protein.

In some embodiments, the engineered cell comprises a chimeric antigen binding receptor comprising an extracellular target-binding moiety that binds EIIIB fibronectin and an intracellular signaling domain, and secretes a VHH or VHH fusion protein that binds PD-L1. In some embodiments, the engineered cell comprises a chimeric antigen binding receptor comprising an extracellular target-binding moiety that is a an anti-EIIIB fibronectin VHH (e.g., B2 or variants thereof) and an intracellular signaling domain, and secretes an anti-PD-L1 VHH (e.g., A12, B3, or variants thereof) or an anti-PD-L1 VHH fusion protein (e.g., A12-Fc, B3-Fc, or variants thereof).

In some embodiments, the engineered cell comprises a chimeric antigen binding receptor comprising an extracellular target-binding moiety that binds EIIIB fibronectin and an intracellular signaling domain, and secretes a VHH or VHH fusion protein that binds TIM3. In some embodiments, the engineered cell comprises a chimeric antigen binding receptor comprising an extracellular target-binding moiety that is a an anti-EIIIB fibronectin VHH (e.g., B2 or variants thereof) and an intracellular signaling domain, and secretes an anti-TIM3 VHH (e.g., mH2, hH6 or variants thereof) or an anti-TIM3 VHH fusion protein (e.g., mH2-Fc, hH6-Fcor variants thereof).

In some embodiments, the engineered cell comprises a chimeric antigen binding receptor comprising an extracellular target-binding moiety that binds EIIIB fibronectin and an intracellular signaling domain, and secretes a VHH or VHH fusion protein that binds LAG3. In some embodiments, the engineered cell comprises a chimeric antigen binding receptor comprising an extracellular target-binding moiety that is a an anti-EIIIB fibronectin VHH (e.g., B2 or variants thereof) and an intracellular signaling domain, and secretes an anti-LAG3 VHH or an anti-LAG3 VHH fusion protein (e.g., Fc fusion or variants thereof).

In some embodiments, the engineered cell comprises a chimeric antigen binding receptor comprising an extracellular target-binding moiety that binds EIIIB fibronectin and an intracellular signaling domain, and secretes a VHH or VHH fusion protein that binds TIM3 and LAG3. In some embodiments, the engineered cell comprises a chimeric antigen binding receptor comprising an extracellular target-binding moiety that is a an anti-EIIIB fibronectin VHH (e.g., B2 or variants thereof) and an intracellular signaling domain, and secretes a bispecific VHH comprising an anti-TIM3 VHH (e.g., mH2, hH6 or variants thereof) fused to an anti-LAG3 VHH or variants thereof.

In some embodiments, the engineered cell comprises a chimeric antigen binding receptor comprising an extracellular target-binding moiety that binds PD-L1 and an intracellular signaling domain, and secretes a VHH or VHH fusion protein that binds LAG3.

In some embodiments, the engineered cell comprises a chimeric antigen binding receptor comprising an extracellular target-binding moiety that binds PD-L1 and an intracellular signaling domain, and secretes a VHH or VHH fusion protein that binds VISTA.

In some embodiments, the engineered cell comprises a chimeric antigen binding receptor comprising an extracellular target-binding moiety that binds PD-L1 and an intracellular signaling domain, and secretes a VHH or VHH fusion protein that binds Siglec15.

In some embodiments, the engineered cell comprises a chimeric antigen binding receptor comprising an extracellular target-binding moiety that binds PD-L1 and an intracellular signaling domain, and secretes a VHH or VHH fusion protein that binds VEGFR.

In some embodiments, the engineered cell comprises a chimeric antigen binding receptor comprising an extracellular target-binding moiety that binds PD-L1 and an intracellular signaling domain, and secretes a VHH or VHH fusion protein that binds HER2.

In some embodiments, the engineered cell comprises a chimeric antigen binding receptor comprising an extracellular target-binding moiety that binds PD-L1 and an intracellular signaling domain, and secretes a VHH or VHH fusion protein that binds PSMA.

In some embodiments, the engineered cell comprises a chimeric antigen binding receptor comprising an extracellular target-binding moiety that binds PD-L1 and an intracellular signaling domain, and secretes a VHH or VHH fusion protein that binds AXL.

In some embodiments, the engineered cell comprises a chimeric antigen binding receptor comprising an extracellular target-binding moiety that binds PD-L1 and an intracellular signaling domain, and secretes a VHH or VHH fusion protein that binds Muc1.

In some embodiments, the engineered cell comprises a chimeric antigen binding receptor comprising an extracellular target-binding moiety that binds PD-L1 and an intracellular signaling domain, and secretes a VHH or VHH fusion protein that binds Muc16.

In some embodiments, the engineered cell comprises a chimeric antigen binding receptor comprising an extracellular target-binding moiety that binds PD-L1 and an intracellular signaling domain, and secretes a VHH or VHH fusion protein that binds MHCI/II (e.g., VHH7, VHH7-Fc fusion, or variants thereof).

In some embodiments, the engineered cell comprises a chimeric antigen binding receptor comprising an extracellular target-binding moiety that binds EIIIB FIBRONECTIN and an intracellular signaling domain, and secretes a VHH or VHH fusion protein that binds LAGS.

In some embodiments, the engineered cell comprises a chimeric antigen binding receptor comprising an extracellular target-binding moiety that binds EIIIB FIBRONECTIN and an intracellular signaling domain, and secretes a VHH or VHH fusion protein that binds VISTA.

In some embodiments, the engineered cell comprises a chimeric antigen binding receptor comprising an extracellular target-binding moiety that binds EIIIB FIBRONECTIN and an intracellular signaling domain, and secretes a VHH or VHH fusion protein that binds Siglec15.

In some embodiments, the engineered cell comprises a chimeric antigen binding receptor comprising an extracellular target-binding moiety that binds EIIIB FIBRONECTIN and an intracellular signaling domain, and secretes a VHH or VHH fusion protein that binds VEGFR.

In some embodiments, the engineered cell comprises a chimeric antigen binding receptor comprising an extracellular target-binding moiety that binds EIIIB FIBRONECTIN and an intracellular signaling domain, and secretes a VHH or VHH fusion protein that binds HER2.

In some embodiments, the engineered cell comprises a chimeric antigen binding receptor comprising an extracellular target-binding moiety that binds EIIIB FIBRONECTIN and an intracellular signaling domain, and secretes a VHH or VHH fusion protein that binds PSMA.

In some embodiments, the engineered cell comprises a chimeric antigen binding receptor comprising an extracellular target-binding moiety that binds EIIIB FIBRONECTIN and an intracellular signaling domain, and secretes a VHH or VHH fusion protein that binds AXL.

In some embodiments, the engineered cell comprises a chimeric antigen binding receptor comprising an extracellular target-binding moiety that binds EIIIB FIBRONECTIN and an intracellular signaling domain, and secretes a VHH or VHH fusion protein that binds Muc1.

In some embodiments, the engineered cell comprises a chimeric antigen binding receptor comprising an extracellular target-binding moiety that binds EIIIB FIBRONECTIN and an intracellular signaling domain, and secretes a VHH or VHH fusion protein that binds Muc16.

In some embodiments, the engineered cell comprises a chimeric antigen binding receptor comprising an extracellular target-binding moiety that binds EIIIB FIBRONECTIN and an intracellular signaling domain, and secretes a VHH or VHH fusion protein that binds MHCI/II (e.g., VHH7, VHH7-Fc fusion, or variants thereof).

In some embodiments, the engineered cell comprises a chimeric antigen binding receptor comprising an extracellular target-binding moiety that binds CEA and an intracellular signaling domain, and secretes a VHH or VHH fusion protein that binds CD47.

In some embodiments, the engineered cell comprises a chimeric antigen binding receptor comprising an extracellular target-binding moiety that binds CEA and an intracellular signaling domain, and secretes a VHH or VHH fusion protein that binds CTLA4.

In some embodiments, the engineered cell comprises a chimeric antigen binding receptor comprising an extracellular target-binding moiety that binds CEA and an intracellular signaling domain, and secretes a VHH or VHH fusion protein that binds PD-1.

In some embodiments, the engineered cell comprises a chimeric antigen binding receptor comprising an extracellular target-binding moiety that binds CEA and an intracellular signaling domain, and secretes a VHH or VHH fusion protein that binds PD-L1.

In some embodiments, the engineered cell comprises a chimeric antigen binding receptor comprising an extracellular target-binding moiety that binds CEA and an intracellular signaling domain, and secretes a VHH or VHH fusion protein that binds TIM3.

In some embodiments, the engineered cell comprises a chimeric antigen binding receptor comprising an extracellular target-binding moiety that binds CEA and an intracellular signaling domain, and secretes a VHH or VHH fusion protein that binds EIIB fibronectin.

In some embodiments, the engineered cell comprises a chimeric antigen binding receptor comprising an extracellular target-binding moiety that binds CEA and an intracellular signaling domain, and secretes a VHH or VHH fusion protein that binds LAGS.

In some embodiments, the engineered cell comprises a chimeric antigen binding receptor comprising an extracellular target-binding moiety that binds CEA and an intracellular signaling domain, and secretes a VHH or VHH fusion protein that binds VISTA.

In some embodiments, the engineered cell comprises a chimeric antigen binding receptor comprising an extracellular target-binding moiety that binds CEA and an intracellular signaling domain, and secretes a VHH or VHH fusion protein that binds Siglec15.

In some embodiments, the engineered cell comprises a chimeric antigen binding receptor comprising an extracellular target-binding moiety that binds CEA and an intracellular signaling domain, and secretes a VHH or VHH fusion protein that binds VEGFR.

In some embodiments, the engineered cell comprises a chimeric antigen binding receptor comprising an extracellular target-binding moiety that binds CEA and an intracellular signaling domain, and secretes a VHH or VHH fusion protein that binds HER2.

In some embodiments, the engineered cell comprises a chimeric antigen binding receptor comprising an extracellular target-binding moiety that binds CEA and an intracellular signaling domain, and secretes a VHH or VHH fusion protein that binds PSMA.

In some embodiments, the engineered cell comprises a chimeric antigen binding receptor comprising an extracellular target-binding moiety that binds CEA and an intracellular signaling domain, and secretes a VHH or VHH fusion protein that binds AXL.

In some embodiments, the engineered cell comprises a chimeric antigen binding receptor comprising an extracellular target-binding moiety that binds CEA and an intracellular signaling domain, and secretes a VHH or VHH fusion protein that binds Muc1.

In some embodiments, the engineered cell comprises a chimeric antigen binding receptor comprising an extracellular target-binding moiety that binds CEA and an intracellular signaling domain, and secretes a VHH or VHH fusion protein that binds Muc16.

In some embodiments, the engineered cell comprises a chimeric antigen binding receptor comprising an extracellular target-binding moiety that binds CEA and an intracellular signaling domain, and secretes a VHH or VHH fusion protein that binds MHCI/II.

In some embodiments, the engineered cell comprises a chimeric antigen binding receptor comprising an extracellular target-binding moiety that binds PSMA and an intracellular signaling domain, and secretes a VHH or VHH fusion protein that binds CD47.

In some embodiments, the engineered cell comprises a chimeric antigen binding receptor comprising an extracellular target-binding moiety that binds PSMA and an intracellular signaling domain, and secretes a VHH or VHH fusion protein that binds CTLA4.

In some embodiments, the engineered cell comprises a chimeric antigen binding receptor comprising an extracellular target-binding moiety that binds PSMA and an intracellular signaling domain, and secretes a VHH or VHH fusion protein that binds PD-1.

In some embodiments, the engineered cell comprises a chimeric antigen binding receptor comprising an extracellular target-binding moiety that binds PSMA and an intracellular signaling domain, and secretes a VHH or VHH fusion protein that binds PD-L1.

In some embodiments, the engineered cell comprises a chimeric antigen binding receptor comprising an extracellular target-binding moiety that binds PSMA and an intracellular signaling domain, and secretes a VHH or VHH fusion protein that binds TIM3.

In some embodiments, the engineered cell comprises a chimeric antigen binding receptor comprising an extracellular target-binding moiety that binds PSMA and an intracellular signaling domain, and secretes a VHH or VHH fusion protein that binds EIIB fibronectin.

In some embodiments, the engineered cell comprises a chimeric antigen binding receptor comprising an extracellular target-binding moiety that binds PSMA and an intracellular signaling domain, and secretes a VHH or VHH fusion protein that binds LAG3.

In some embodiments, the engineered cell comprises a chimeric antigen binding receptor comprising an extracellular target-binding moiety that binds PSMA and an intracellular signaling domain, and secretes a VHH or VHH fusion protein that binds VISTA.

In some embodiments, the engineered cell comprises a chimeric antigen binding receptor comprising an extracellular target-binding moiety that binds PSMA and an intracellular signaling domain, and secretes a VHH or VHH fusion protein that binds Siglec15.

In some embodiments, the engineered cell comprises a chimeric antigen binding receptor comprising an extracellular target-binding moiety that binds PSMA and an intracellular signaling domain, and secretes a VHH or VHH fusion protein that binds VEGFR.

In some embodiments, the engineered cell comprises a chimeric antigen binding receptor comprising an extracellular target-binding moiety that binds PSMA and an intracellular signaling domain, and secretes a VHH or VHH fusion protein that binds HER2.

In some embodiments, the engineered cell comprises a chimeric antigen binding receptor comprising an extracellular target-binding moiety that binds PSMA and an intracellular signaling domain, and secretes a VHH or VHH fusion protein that binds PSMA.

In some embodiments, the engineered cell comprises a chimeric antigen binding receptor comprising an extracellular target-binding moiety that binds PSMA and an intracellular signaling domain, and secretes a VHH or VHH fusion protein that binds AXL.

In some embodiments, the engineered cell comprises a chimeric antigen binding receptor comprising an extracellular target-binding moiety that binds PSMA and an intracellular signaling domain, and secretes a VHH or VHH fusion protein that binds Muc1.

In some embodiments, the engineered cell comprises a chimeric antigen binding receptor comprising an extracellular target-binding moiety that binds PSMA and an intracellular signaling domain, and secretes a VHH or VHH fusion protein that binds Muc16.

In some embodiments, the engineered cell comprises a chimeric antigen binding receptor comprising an extracellular target-binding moiety that binds PSMA and an intracellular signaling domain, and secretes a VHH or VHH fusion protein that binds MHCI/II.

In some embodiments, the engineered cell comprises a chimeric antigen binding receptor comprising an extracellular target-binding moiety that binds AXL and an intracellular signaling domain, and secretes a VHH or VHH fusion protein that binds CD47.

In some embodiments, the engineered cell comprises a chimeric antigen binding receptor comprising an extracellular target-binding moiety that binds AXL and an intracellular signaling domain, and secretes a VHH or VHH fusion protein that binds CTLA4.

In some embodiments, the engineered cell comprises a chimeric antigen binding receptor comprising an extracellular target-binding moiety that binds AXL and an intracellular signaling domain, and secretes a VHH or VHH fusion protein that binds PD-1.

In some embodiments, the engineered cell comprises a chimeric antigen binding receptor comprising an extracellular target-binding moiety that binds AXL and an intracellular signaling domain, and secretes a VHH or VHH fusion protein that binds PD-L1.

In some embodiments, the engineered cell comprises a chimeric antigen binding receptor comprising an extracellular target-binding moiety that binds AXL and an intracellular signaling domain, and secretes a VHH or VHH fusion protein that binds TIM3.

In some embodiments, the engineered cell comprises a chimeric antigen binding receptor comprising an extracellular target-binding moiety that binds AXL and an intracellular signaling domain, and secretes a VHH or VHH fusion protein that binds EIIB fibronectin.

In some embodiments, the engineered cell comprises a chimeric antigen binding receptor comprising an extracellular target-binding moiety that binds AXL and an intracellular signaling domain, and secretes a VHH or VHH fusion protein that binds LAGS.

In some embodiments, the engineered cell comprises a chimeric antigen binding receptor comprising an extracellular target-binding moiety that binds AXL and an intracellular signaling domain, and secretes a VHH or VHH fusion protein that binds VISTA.

In some embodiments, the engineered cell comprises a chimeric antigen binding receptor comprising an extracellular target-binding moiety that binds AXL and an intracellular signaling domain, and secretes a VHH or VHH fusion protein that binds Siglec15.

In some embodiments, the engineered cell comprises a chimeric antigen binding receptor comprising an extracellular target-binding moiety that binds AXL and an intracellular signaling domain, and secretes a VHH or VHH fusion protein that binds VEGFR.

In some embodiments, the engineered cell comprises a chimeric antigen binding receptor comprising an extracellular target-binding moiety that binds AXL and an intracellular signaling domain, and secretes a VHH or VHH fusion protein that binds HER2.

In some embodiments, the engineered cell comprises a chimeric antigen binding receptor comprising an extracellular target-binding moiety that binds AXL and an intracellular signaling domain, and secretes a VHH or VHH fusion protein that binds PSMA.

In some embodiments, the engineered cell comprises a chimeric antigen binding receptor comprising an extracellular target-binding moiety that binds AXL and an intracellular signaling domain, and secretes a VHH or VHH fusion protein that binds AXL.

In some embodiments, the engineered cell comprises a chimeric antigen binding receptor comprising an extracellular target-binding moiety that binds AXL and an intracellular signaling domain, and secretes a VHH or VHH fusion protein that binds Muc1.

In some embodiments, the engineered cell comprises a chimeric antigen binding receptor comprising an extracellular target-binding moiety that binds AXL and an intracellular signaling domain, and secretes a VHH or VHH fusion protein that binds Muc16.

In some embodiments, the engineered cell comprises a chimeric antigen binding receptor comprising an extracellular target-binding moiety that binds AXL and an intracellular signaling domain, and secretes a VHH or VHH fusion protein that binds MHCI/II.

In some embodiments, the engineered cell comprises a chimeric antigen binding receptor comprising an extracellular target-binding moiety that binds HER2 and an intracellular signaling domain, and secretes a VHH or VHH fusion protein that binds CD47.

In some embodiments, the engineered cell comprises a chimeric antigen binding receptor comprising an extracellular target-binding moiety that binds HER2 and an intracellular signaling domain, and secretes a VHH or VHH fusion protein that binds CTLA4.

In some embodiments, the engineered cell comprises a chimeric antigen binding receptor comprising an extracellular target-binding moiety that binds HER2 and an intracellular signaling domain, and secretes a VHH or VHH fusion protein that binds PD-1.

In some embodiments, the engineered cell comprises a chimeric antigen binding receptor comprising an extracellular target-binding moiety that binds HER2 and an intracellular signaling domain, and secretes a VHH or VHH fusion protein that binds PD-L1.

In some embodiments, the engineered cell comprises a chimeric antigen binding receptor comprising an extracellular target-binding moiety that binds HER2 and an intracellular signaling domain, and secretes a VHH or VHH fusion protein that binds TIM3.

In some embodiments, the engineered cell comprises a chimeric antigen binding receptor comprising an extracellular target-binding moiety that binds HER2 and an intracellular signaling domain, and secretes a VHH or VHH fusion protein that binds EIIB fibronectin.

In some embodiments, the engineered cell comprises a chimeric antigen binding receptor comprising an extracellular target-binding moiety that binds HER2 and an intracellular signaling domain, and secretes a VHH or VHH fusion protein that binds LAG3.

In some embodiments, the engineered cell comprises a chimeric antigen binding receptor comprising an extracellular target-binding moiety that binds HER2 and an intracellular signaling domain, and secretes a VHH or VHH fusion protein that binds VISTA.

In some embodiments, the engineered cell comprises a chimeric antigen binding receptor comprising an extracellular target-binding moiety that binds HER2 and an intracellular signaling domain, and secretes a VHH or VHH fusion protein that binds Siglec15.

In some embodiments, the engineered cell comprises a chimeric antigen binding receptor comprising an extracellular target-binding moiety that binds HER2 and an intracellular signaling domain, and secretes a VHH or VHH fusion protein that binds VEGFR.

In some embodiments, the engineered cell comprises a chimeric antigen binding receptor comprising an extracellular target-binding moiety that binds HER2 and an intracellular signaling domain, and secretes a VHH or VHH fusion protein that binds HER2.

In some embodiments, the engineered cell comprises a chimeric antigen binding receptor comprising an extracellular target-binding moiety that binds HER2 and an intracellular signaling domain, and secretes a VHH or VHH fusion protein that binds PSMA.

In some embodiments, the engineered cell comprises a chimeric antigen binding receptor comprising an extracellular target-binding moiety that binds HER2 and an intracellular signaling domain, and secretes a VHH or VHH fusion protein that binds HER2.

In some embodiments, the engineered cell comprises a chimeric antigen binding receptor comprising an extracellular target-binding moiety that binds HER2 and an intracellular signaling domain, and secretes a VHH or VHH fusion protein that binds Muc1.

In some embodiments, the engineered cell comprises a chimeric antigen binding receptor comprising an extracellular target-binding moiety that binds HER2 and an intracellular signaling domain, and secretes a VHH or VHH fusion protein that binds Muc16.

In some embodiments, the engineered cell comprises a chimeric antigen binding receptor comprising an extracellular target-binding moiety that binds HER2 and an intracellular signaling domain, and secretes a VHH or VHH fusion protein that binds MHCI/II.

In some embodiments, the engineered cell comprises a chimeric antigen binding receptor comprising an extracellular target-binding moiety that binds CD133 and an intracellular signaling domain, and secretes a VHH or VHH fusion protein that binds CD47.

In some embodiments, the engineered cell comprises a chimeric antigen binding receptor comprising an extracellular target-binding moiety that binds CD133 and an intracellular signaling domain, and secretes a VHH or VHH fusion protein that binds CTLA4.

In some embodiments, the engineered cell comprises a chimeric antigen binding receptor comprising an extracellular target-binding moiety that binds CD133 and an intracellular signaling domain, and secretes a VHH or VHH fusion protein that binds PD-1.

In some embodiments, the engineered cell comprises a chimeric antigen binding receptor comprising an extracellular target-binding moiety that binds CD133 and an intracellular signaling domain, and secretes a VHH or VHH fusion protein that binds PD-L1.

In some embodiments, the engineered cell comprises a chimeric antigen binding receptor comprising an extracellular target-binding moiety that binds CD133 and an intracellular signaling domain, and secretes a VHH or VHH fusion protein that binds TIM3.

In some embodiments, the engineered cell comprises a chimeric antigen binding receptor comprising an extracellular target-binding moiety that binds CD133 and an intracellular signaling domain, and secretes a VHH or VHH fusion protein that binds EIIB fibronectin.

In some embodiments, the engineered cell comprises a chimeric antigen binding receptor comprising an extracellular target-binding moiety that binds CD133 and an intracellular signaling domain, and secretes a VHH or VHH fusion protein that binds LAG3.

In some embodiments, the engineered cell comprises a chimeric antigen binding receptor comprising an extracellular target-binding moiety that binds CD133 and an intracellular signaling domain, and secretes a VHH or VHH fusion protein that binds VISTA.

In some embodiments, the engineered cell comprises a chimeric antigen binding receptor comprising an extracellular target-binding moiety that binds CD133 and an intracellular signaling domain, and secretes a VHH or VHH fusion protein that binds Siglec15.

In some embodiments, the engineered cell comprises a chimeric antigen binding receptor comprising an extracellular target-binding moiety that binds CD133 and an intracellular signaling domain, and secretes a VHH or VHH fusion protein that binds VEGFR.

In some embodiments, the engineered cell comprises a chimeric antigen binding receptor comprising an extracellular target-binding moiety that binds CD133 and an intracellular signaling domain, and secretes a VHH or VHH fusion protein that binds HER2.

In some embodiments, the engineered cell comprises a chimeric antigen binding receptor comprising an extracellular target-binding moiety that binds CD133 and an intracellular signaling domain, and secretes a VHH or VHH fusion protein that binds PSMA.

In some embodiments, the engineered cell comprises a chimeric antigen binding receptor comprising an extracellular target-binding moiety that binds CD133 and an intracellular signaling domain, and secretes a VHH or VHH fusion protein that binds CD133.

In some embodiments, the engineered cell comprises a chimeric antigen binding receptor comprising an extracellular target-binding moiety that binds CD133 and an intracellular signaling domain, and secretes a VHH or VHH fusion protein that binds Muc1.

In some embodiments, the engineered cell comprises a chimeric antigen binding receptor comprising an extracellular target-binding moiety that binds CD133 and an intracellular signaling domain, and secretes a VHH or VHH fusion protein that binds Muc16.

In some embodiments, the engineered cell comprises a chimeric antigen binding receptor comprising an extracellular target-binding moiety that binds CD133 and an intracellular signaling domain, and secretes a VHH or VHH fusion protein that binds MHCI/II.

In some embodiments, the engineered cell comprises a chimeric antigen binding receptor comprising an extracellular target-binding moiety that binds MUC1 and an intracellular signaling domain, and secretes a VHH or VHH fusion protein that binds CD47.

In some embodiments, the engineered cell comprises a chimeric antigen binding receptor comprising an extracellular target-binding moiety that binds MUC1 and an intracellular signaling domain, and secretes a VHH or VHH fusion protein that binds CTLA4.

In some embodiments, the engineered cell comprises a chimeric antigen binding receptor comprising an extracellular target-binding moiety that binds MUC1 and an intracellular signaling domain, and secretes a VHH or VHH fusion protein that binds PD-1.

In some embodiments, the engineered cell comprises a chimeric antigen binding receptor comprising an extracellular target-binding moiety that binds MUC1 and an intracellular signaling domain, and secretes a VHH or VHH fusion protein that binds PD-L1.

In some embodiments, the engineered cell comprises a chimeric antigen binding receptor comprising an extracellular target-binding moiety that binds MUC1 and an intracellular signaling domain, and secretes a VHH or VHH fusion protein that binds TIM3.

In some embodiments, the engineered cell comprises a chimeric antigen binding receptor comprising an extracellular target-binding moiety that binds MUC1 and an intracellular signaling domain, and secretes a VHH or VHH fusion protein that binds EIIB fibronectin.

In some embodiments, the engineered cell comprises a chimeric antigen binding receptor comprising an extracellular target-binding moiety that binds MUC1 and an intracellular signaling domain, and secretes a VHH or VHH fusion protein that binds LAG3.

In some embodiments, the engineered cell comprises a chimeric antigen binding receptor comprising an extracellular target-binding moiety that binds MUC1 and an intracellular signaling domain, and secretes a VHH or VHH fusion protein that binds VISTA.

In some embodiments, the engineered cell comprises a chimeric antigen binding receptor comprising an extracellular target-binding moiety that binds MUC1 and an intracellular signaling domain, and secretes a VHH or VHH fusion protein that binds Siglec15.

In some embodiments, the engineered cell comprises a chimeric antigen binding receptor comprising an extracellular target-binding moiety that binds MUC1 and an intracellular signaling domain, and secretes a VHH or VHH fusion protein that binds VEGFR.

In some embodiments, the engineered cell comprises a chimeric antigen binding receptor comprising an extracellular target-binding moiety that binds MUC1 and an intracellular signaling domain, and secretes a VHH or VHH fusion protein that binds HER2.

In some embodiments, the engineered cell comprises a chimeric antigen binding receptor comprising an extracellular target-binding moiety that binds MUC1 and an intracellular signaling domain, and secretes a VHH or VHH fusion protein that binds PSMA.

In some embodiments, the engineered cell comprises a chimeric antigen binding receptor comprising an extracellular target-binding moiety that binds MUC1 and an intracellular signaling domain, and secretes a VHH or VHH fusion protein that binds CD133.

In some embodiments, the engineered cell comprises a chimeric antigen binding receptor comprising an extracellular target-binding moiety that binds MUC1 and an intracellular signaling domain, and secretes a VHH or VHH fusion protein that binds Muc1.

In some embodiments, the engineered cell comprises a chimeric antigen binding receptor comprising an extracellular target-binding moiety that binds MUC1 and an intracellular signaling domain, and secretes a VHH or VHH fusion protein that binds Muc16.

In some embodiments, the engineered cell comprises a chimeric antigen binding receptor comprising an extracellular target-binding moiety that binds MUC1 and an intracellular signaling domain, and secretes a VHH or VHH fusion protein that binds MHCI/II.

In some embodiments, the engineered cell comprises a chimeric antigen binding receptor comprising an extracellular target-binding moiety that binds MUC16 and an intracellular signaling domain, and secretes a VHH or VHH fusion protein that binds CD47.

In some embodiments, the engineered cell comprises a chimeric antigen binding receptor comprising an extracellular target-binding moiety that binds MUC16 and an intracellular signaling domain, and secretes a VHH or VHH fusion protein that binds CTLA4.

In some embodiments, the engineered cell comprises a chimeric antigen binding receptor comprising an extracellular target-binding moiety that binds MUC16 and an intracellular signaling domain, and secretes a VHH or VHH fusion protein that binds PD-1.

In some embodiments, the engineered cell comprises a chimeric antigen binding receptor comprising an extracellular target-binding moiety that binds MUC16 and an intracellular signaling domain, and secretes a VHH or VHH fusion protein that binds PD-L1.

In some embodiments, the engineered cell comprises a chimeric antigen binding receptor comprising an extracellular target-binding moiety that binds MUC16 and an intracellular signaling domain, and secretes a VHH or VHH fusion protein that binds TIM3.

In some embodiments, the engineered cell comprises a chimeric antigen binding receptor comprising an extracellular target-binding moiety that binds MUC16 and an intracellular signaling domain, and secretes a VHH or VHH fusion protein that binds EIIB fibronectin.

In some embodiments, the engineered cell comprises a chimeric antigen binding receptor comprising an extracellular target-binding moiety that binds MUC16 and an intracellular signaling domain, and secretes a VHH or VHH fusion protein that binds LAG3.

In some embodiments, the engineered cell comprises a chimeric antigen binding receptor comprising an extracellular target-binding moiety that binds MUC16 and an intracellular signaling domain, and secretes a VHH or VHH fusion protein that binds VISTA.

In some embodiments, the engineered cell comprises a chimeric antigen binding receptor comprising an extracellular target-binding moiety that binds MUC16 and an intracellular signaling domain, and secretes a VHH or VHH fusion protein that binds Siglec15.

In some embodiments, the engineered cell comprises a chimeric antigen binding receptor comprising an extracellular target-binding moiety that binds MUC16 and an intracellular signaling domain, and secretes a VHH or VHH fusion protein that binds VEGFR.

In some embodiments, the engineered cell comprises a chimeric antigen binding receptor comprising an extracellular target-binding moiety that binds MUC16 and an intracellular signaling domain, and secretes a VHH or VHH fusion protein that binds HER2.

In some embodiments, the engineered cell comprises a chimeric antigen binding receptor comprising an extracellular target-binding moiety that binds MUC16 and an intracellular signaling domain, and secretes a VHH or VHH fusion protein that binds PSMA.

In some embodiments, the engineered cell comprises a chimeric antigen binding receptor comprising an extracellular target-binding moiety that binds MUC16 and an intracellular signaling domain, and secretes a VHH or VHH fusion protein that binds CD133.

In some embodiments, the engineered cell comprises a chimeric antigen binding receptor comprising an extracellular target-binding moiety that binds MUC16 and an intracellular signaling domain, and secretes a VHH or VHH fusion protein that binds Muc1.

In some embodiments, the engineered cell comprises a chimeric antigen binding receptor comprising an extracellular target-binding moiety that binds MUC16 and an intracellular signaling domain, and secretes a VHH or VHH fusion protein that binds Muc16.

In some embodiments, the engineered cell comprises a chimeric antigen binding receptor comprising an extracellular target-binding moiety that binds MUC16 and an intracellular signaling domain, and secretes a VHH or VHH fusion protein that binds MHCI/II.

In some embodiments, the engineered cell comprises a chimeric antigen binding receptor comprising an extracellular target-binding moiety that binds SIGLEC15 and an intracellular signaling domain, and secretes a VHH or VHH fusion protein that binds CD47.

In some embodiments, the engineered cell comprises a chimeric antigen binding receptor comprising an extracellular target-binding moiety that binds SIGLEC15 and an intracellular signaling domain, and secretes a VHH or VHH fusion protein that binds CTLA4.

In some embodiments, the engineered cell comprises a chimeric antigen binding receptor comprising an extracellular target-binding moiety that binds SIGLEC15 and an intracellular signaling domain, and secretes a VHH or VHH fusion protein that binds PD-1.

In some embodiments, the engineered cell comprises a chimeric antigen binding receptor comprising an extracellular target-binding moiety that binds SIGLEC15 and an intracellular signaling domain, and secretes a VHH or VHH fusion protein that binds PD-L1.

In some embodiments, the engineered cell comprises a chimeric antigen binding receptor comprising an extracellular target-binding moiety that binds SIGLEC15 and an intracellular signaling domain, and secretes a VHH or VHH fusion protein that binds TIM3.

In some embodiments, the engineered cell comprises a chimeric antigen binding receptor comprising an extracellular target-binding moiety that binds SIGLEC15 and an intracellular signaling domain, and secretes a VHH or VHH fusion protein that binds EIIB fibronectin.

In some embodiments, the engineered cell comprises a chimeric antigen binding receptor comprising an extracellular target-binding moiety that binds SIGLEC15 and an intracellular signaling domain, and secretes a VHH or VHH fusion protein that binds LAG3.

In some embodiments, the engineered cell comprises a chimeric antigen binding receptor comprising an extracellular target-binding moiety that binds SIGLEC15 and an intracellular signaling domain, and secretes a VHH or VHH fusion protein that binds VISTA.

In some embodiments, the engineered cell comprises a chimeric antigen binding receptor comprising an extracellular target-binding moiety that binds SIGLEC15 and an intracellular signaling domain, and secretes a VHH or VHH fusion protein that binds Siglec15.

In some embodiments, the engineered cell comprises a chimeric antigen binding receptor comprising an extracellular target-binding moiety that binds SIGLEC15 and an intracellular signaling domain, and secretes a VHH or VHH fusion protein that binds VEGFR.

In some embodiments, the engineered cell comprises a chimeric antigen binding receptor comprising an extracellular target-binding moiety that binds SIGLEC15 and an intracellular signaling domain, and secretes a VHH or VHH fusion protein that binds HER2.

In some embodiments, the engineered cell comprises a chimeric antigen binding receptor comprising an extracellular target-binding moiety that binds SIGLEC15 and an intracellular signaling domain, and secretes a VHH or VHH fusion protein that binds PSMA.

In some embodiments, the engineered cell comprises a chimeric antigen binding receptor comprising an extracellular target-binding moiety that binds SIGLEC15 and an intracellular signaling domain, and secretes a VHH or VHH fusion protein that binds CD133.

In some embodiments, the engineered cell comprises a chimeric antigen binding receptor comprising an extracellular target-binding moiety that binds SIGLEC15 and an intracellular signaling domain, and secretes a VHH or VHH fusion protein that binds Muc1.

In some embodiments, the engineered cell comprises a chimeric antigen binding receptor comprising an extracellular target-binding moiety that binds SIGLEC15 and an intracellular signaling domain, and secretes a VHH or VHH fusion protein that binds Muc16.

In some embodiments, the engineered cell comprises a chimeric antigen binding receptor comprising an extracellular target-binding moiety that binds SIGLEC15 and an intracellular signaling domain, and secretes a VHH or VHH fusion protein that binds MHCI/II.

In some embodiments, the engineered cell comprises a chimeric antigen binding receptor comprising an extracellular target-binding moiety that binds mesothelin and an intracellular signaling domain, and secretes a VHH or VHH fusion protein that binds CD47.

In some embodiments, the engineered cell comprises a chimeric antigen binding receptor comprising an extracellular target-binding moiety that binds mesothelin and an intracellular signaling domain, and secretes a VHH or VHH fusion protein that binds CTLA4.

In some embodiments, the engineered cell comprises a chimeric antigen binding receptor comprising an extracellular target-binding moiety that binds mesothelin and an intracellular signaling domain, and secretes a VHH or VHH fusion protein that binds PD-1.

In some embodiments, the engineered cell comprises a chimeric antigen binding receptor comprising an extracellular target-binding moiety that binds mesothelin and an intracellular signaling domain, and secretes a VHH or VHH fusion protein that binds PD-L1.

In some embodiments, the engineered cell comprises a chimeric antigen binding receptor comprising an extracellular target-binding moiety that binds mesothelin and an intracellular signaling domain, and secretes a VHH or VHH fusion protein that binds TIM3.

In some embodiments, the engineered cell comprises a chimeric antigen binding receptor comprising an extracellular target-binding moiety that binds mesothelin and an intracellular signaling domain, and secretes a VHH or VHH fusion protein that binds EIIB fibronectin.

In some embodiments, the engineered cell comprises a chimeric antigen binding receptor comprising an extracellular target-binding moiety that binds mesothelin and an intracellular signaling domain, and secretes a VHH or VHH fusion protein that binds LAG3.

In some embodiments, the engineered cell comprises a chimeric antigen binding receptor comprising an extracellular target-binding moiety that binds mesothelin and an intracellular signaling domain, and secretes a VHH or VHH fusion protein that binds VISTA.

In some embodiments, the engineered cell comprises a chimeric antigen binding receptor comprising an extracellular target-binding moiety that binds mesothelin and an intracellular signaling domain, and secretes a VHH or VHH fusion protein that binds Siglec15.

In some embodiments, the engineered cell comprises a chimeric antigen binding receptor comprising an extracellular target-binding moiety that binds mesothelin and an intracellular signaling domain, and secretes a VHH or VHH fusion protein that binds VEGFR.

In some embodiments, the engineered cell comprises a chimeric antigen binding receptor comprising an extracellular target-binding moiety that binds mesothelin and an intracellular signaling domain, and secretes a VHH or VHH fusion protein that binds HER2.

In some embodiments, the engineered cell comprises a chimeric antigen binding receptor comprising an extracellular target-binding moiety that binds mesothelin and an intracellular signaling domain, and secretes a VHH or VHH fusion protein that binds PSMA.

In some embodiments, the engineered cell comprises a chimeric antigen binding receptor comprising an extracellular target-binding moiety that binds mesothelin and an intracellular signaling domain, and secretes a VHH or VHH fusion protein that binds CD133.

In some embodiments, the engineered cell comprises a chimeric antigen binding receptor comprising an extracellular target-binding moiety that binds mesothelin and an intracellular signaling domain, and secretes a VHH or VHH fusion protein that binds Muc1.

In some embodiments, the engineered cell comprises a chimeric antigen binding receptor comprising an extracellular target-binding moiety that binds mesothelin and an intracellular signaling domain, and secretes a VHH or VHH fusion protein that binds Muc16.

In some embodiments, the engineered cell comprises a chimeric antigen binding receptor comprising an extracellular target-binding moiety that binds mesothelin and an intracellular signaling domain, and secretes a VHH or VHH fusion protein that binds MHCI/II.

In some embodiments, the engineered cell comprises a chimeric antigen binding receptor comprising an extracellular target-binding moiety that binds PD-L1 and an intracellular signaling domain, and secretes a VHH or VHH fusion protein that binds an autoimmune antigen selected from antigen-specific TCRs, BCRs, and insulin receptors.

In some embodiments, the engineered cell comprises a chimeric antigen binding receptor comprising an extracellular target-binding moiety that binds EIIB fibronectin and an intracellular signaling domain, and secretes a VHH or VHH fusion protein that binds an autoimmune antigen selected from antigen-specific TCRs, BCRs, and insulin receptors.

In some embodiments, the engineered cell comprises a chimeric antigen binding receptor comprising an extracellular target-binding moiety that binds CEA and an intracellular signaling domain, and secretes a VHH or VHH fusion protein that binds an autoimmune antigen selected from antigen-specific TCRs, BCRs, and insulin receptors.

In some embodiments, the engineered cell comprises a chimeric antigen binding receptor comprising an extracellular target-binding moiety that binds PSMA and an intracellular signaling domain, and secretes a VHH or VHH fusion protein that binds an autoimmune antigen selected from antigen-specific TCRs, BCRs, and insulin receptors.

In some embodiments, the engineered cell comprises a chimeric antigen binding receptor comprising an extracellular target-binding moiety that binds AXL and an intracellular signaling domain, and secretes a VHH or VHH fusion protein that binds an autoimmune antigen selected from antigen-specific TCRs, BCRs, and insulin receptors.

In some embodiments, the engineered cell comprises a chimeric antigen binding receptor comprising an extracellular target-binding moiety that binds HER2 and an intracellular signaling domain, and secretes a VHH or VHH fusion protein that binds an autoimmune antigen selected from antigen-specific TCRs, BCRs, and insulin receptors.

In some embodiments, the engineered cell comprises a chimeric antigen binding receptor comprising an extracellular target-binding moiety that binds CD133 and an intracellular signaling domain, and secretes a VHH or VHH fusion protein that binds an autoimmune antigen selected from antigen-specific TCRs, BCRs, and insulin receptors.

In some embodiments, the engineered cell comprises a chimeric antigen binding receptor comprising an extracellular target-binding moiety that binds Muc1 and an intracellular signaling domain, and secretes a VHH or VHH fusion protein that binds an autoimmune antigen selected from antigen-specific TCRs, BCRs, and insulin receptors.

In some embodiments, the engineered cell comprises a chimeric antigen binding receptor comprising an extracellular target-binding moiety that binds Muc16 and an intracellular signaling domain, and secretes a VHH or VHH fusion protein that binds an autoimmune antigen selected from antigen-specific TCRs, BCRs, and insulin receptors.

In some embodiments, the engineered cell comprises a chimeric antigen binding receptor comprising an extracellular target-binding moiety that binds Siglec15 and an intracellular signaling domain, and secretes a VHH or VHH fusion protein that binds an autoimmune antigen selected from antigen-specific TCRs, BCRs, and insulin receptors.

In some embodiments, the engineered cell comprises a chimeric antigen binding receptor comprising an extracellular target-binding moiety that binds mesothelin and an intracellular signaling domain, and secretes a VHH or VHH fusion protein that binds an autoimmune antigen selected from antigen-specific TCRs, BCRs, and insulin receptors.

The engineered cell of the present disclosure can be an engineered mammalian cell (e.g., human cell). In some embodiments, the engineered cell is an engineered immune cell. An “immune cell” is a cell that plays a role in the immune system. Exemplary immune cells include, without limitation, granulocytes, mast cells, monocytes, neutrophils, dendritic cells, natural killer cells, B cells, T cells including CD4+ T cells, CD8+ T cells, regulatory T cells, and natural killer T cells. In some embodiments, the engineered immune cell is an engineered CD4+ T cell, CD8+ T cell, regulatory T cell, Natural Killer T cell, or Natural Killer cell.

A CD4+ T cell (helper T cell) instigates the adaptive immune responses by recognizing antigen peptides presented on major histocompatibility complex (MHC) Class-II molecules found on antigen presenting cells (APCs).

A CD8+ T cell (cytotoxic T cell) is a T lymphocyte that kills damaged cells, such as cancer cells or infected cells. Damaged cells present MHC Class-I molecules on their cell surface, which are recognized by CD8 T cells, which are then activated to kill the damaged cell.

Regulatory T cells (Treg) are CD4+ T cells which suppress potentially deleterious activities of helper T cells. Among these suppressed activities are: maintaining self-tolerance, suppression of allergy or asthma, suppression of T cell activation triggered by weak stimuli. Regulatory T cells are essential in the activation and growth of cytotoxic T cells.

Natural killer (NK) cells have features of both innate and adaptive immunity. They are important for recognizing and killing virus-infected cells or tumor cells. They contain intracellular compartments called granules, which are filled with proteins that can form holes in the target cell and also cause apoptosis, the process for programmed cell death. It is important to distinguish between apoptosis and other forms of cell death like necrosis. Apoptosis, unlike necrosis, does not release danger signals that can lead to greater immune activation and inflammation. Through apoptosis, immune cells can discreetly remove infected cells and limit bystander damage. Recently, researchers have shown in mouse models that NK cells, like adaptive cells, can be retained as memory cells and respond to subsequent infections by the same pathogen.

Natural killer T (NKT) cells are a heterogeneous group of T cells that share properties of both T cells and natural killer cells. Many of these cells recognize the non-polymorphic CD1d molecule, an antigen-presenting molecule that binds self and foreign lipids and glycolipids.

Other aspects of the present disclosure provide compositions comprising the engineered cell described herein. In some embodiments, the composition is formulated in one or more compositions for administration to the subject. The engineered cell or the composition comprising the engineered cell described herein may be used for the treatment of a disease. As such, methods of treating a disease are also provided, the method comprising administering to a subject in need thereof a therapeutically effective amount of the engineered cell or the composition comprising the cell described herein. In some embodiments, the composition is a pharmaceutical composition. In some embodiments, the composition further comprises a pharmaceutically acceptable carrier.

The term “pharmaceutically-acceptable carrier”, as used herein, means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the agents described herein from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.). Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

In some embodiments, the engineered cell described herein, or composition(s) containing the engineered cell is administered by injection, by means of a catheter, by means of a suppository, or by means of an implant, the implant being of a porous, non-porous, or gelatinous material, including a membrane, such as a sialastic membrane, or a fiber. Typically, when administering the agents or the composition described herein, materials to which the agents does not absorb are used.

In other embodiments, the engineered cell described herein, or composition containing the engineered cell is delivered in a controlled release system. In one embodiment, a pump may be used (see, e.g., Langer, 1990, Science 249:1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et al., 1989, J. Neurosurg. 71:105.) Other controlled release systems are discussed, for example, in Langer, supra.

In some embodiments, the engineered cell described herein, or composition containing the engineered cell is formulated in accordance with routine procedures as a pharmaceutical composition adapted for intravenous or subcutaneous administration to a subject, e.g., a human being. Typically, compositions for administration by injection are solutions in sterile isotonic aqueous buffer. Where necessary, the composition can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the pharmaceutical is administered by injection, an ampoule of sterile water for injection or saline can be provided so that the ingredients can be mixed prior to administration.

A composition for systemic administration may be a liquid, e.g., sterile saline, lactated Ringer's or Hank's solution. In addition, the composition can be in solid forms and re-dissolved or suspended immediately prior to use. Lyophilized forms are also contemplated.

The engineered cell described herein, or composition containing the engineered cell can be contained within a lipid particle or vesicle, such as a liposome or microcrystal, which is also suitable for parenteral administration. The particles can be of any suitable structure, such as unilamellar or plurilamellar, so long as compositions are contained therein. The agents described herein, or composition(s) containing such agents can be entrapped in ‘stabilized plasmid-lipid particles’ (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et al., Gene Ther. 1999, 6:1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757.

The engineered cell described herein, or composition containing the engineered cell of the present disclosure may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the engineered cell described herein, or composition containing the engineered cell can be provided as a pharmaceutical kit comprising (a) a container containing an agent of the disclosure in lyophilized form and (b) a second container containing a pharmaceutically acceptable diluent (e.g., sterile water) for injection. The pharmaceutically acceptable diluent can be used for reconstitution or dilution of the lyophilized agents of the disclosure. Optionally associated with such container(s) can be a notice in the form prescribed by a governmental agency regulating the manufacture, use or sale of pharmaceuticals or biological products, which notice reflects approval by the agency of manufacture, use or sale for human administration.

In some embodiments, an article of manufacture containing materials useful for the treatment of the diseases described above is included. In some embodiments, the article of manufacture comprises a container and a label. Suitable containers include, for example, bottles, vials, syringes, and test tubes. The containers may be formed from a variety of materials such as glass or plastic. In some embodiments, the container holds a composition that is effective for treating a disease described herein and may have a sterile access port. For example, the container may be an intravenous solution bag or a vial having a stopper pierceable by a hypodermic injection needle. The active agent in the composition is an isolated polypeptide of the disclosure. In some embodiments, the label on or associated with the container indicates that the composition is used for treating the disease of choice. The article of manufacture may further comprise a second container comprising a pharmaceutically-acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

The terms “treatment,” “treat,” and “treating” refer to reversing, alleviating, delaying the onset of, or inhibiting the progress of a disease described herein (e.g., cancer or an autoimmune disease). In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence. Prophylactic treatment refers to the treatment of a subject who is not and was not with a disease but is at risk of developing the disease or who was with a disease, is not with the disease, but is at risk of regression of the disease. In some embodiments, the subject is at a higher risk of developing the disease or at a higher risk of regression of the disease than an average healthy member of a population.

An “effective amount” of a composition described herein refers to an amount sufficient to elicit the desired biological response. An effective amount of an agent described herein, or a composition containing such agents may vary depending on such factors as the desired biological endpoint, the pharmacokinetics of the compound, the condition being treated, the mode of administration, and the age and health of the subject. In some embodiments, an effective amount is a therapeutically effective amount. In some embodiments, an effective amount is a prophylactic treatment. In some embodiments, an effective amount is the amount of an agent in a single dose. In some embodiments, an effective amount is the combined amounts of an agent described herein in multiple doses. When an effective amount of a composition is referred herein, it means the amount is prophylactically and/or therapeutically effective, depending on the subject and/or the disease to be treated. Determining the effective amount or dosage is within the abilities of one skilled in the art.

The terms “administer,” “administering,” or “administration” refers to implanting, absorbing, ingesting, injecting, inhaling, or otherwise introducing a compound described herein, or a composition thereof, in or on a subject. The agents described herein, or composition(s) containing such agents may be administered systemically (e.g., via intravenous injection) or locally (e.g., via local injection). In some embodiments, the composition of the vaccine composition described herein is administered via injection, e.g., intravenously, or sublingually. Parenteral administration is also contemplated. The term “parenteral” as used herein includes subcutaneous, intracutaneous, intravenous, intramuscular, intraarticular, intraarterial, intrasynovial, intrasternal, intrathecal, intralesional, intradermally, and intracranial injection or infusion techniques.

Empirical considerations, such as the half-life, generally will contribute to the determination of the dosage. For example, therapeutic agents that are compatible with the human immune system, such as polypeptides comprising regions from humanized antibodies or fully human antibodies, may be used to prolong half-life of the polypeptide and to prevent the polypeptide being attacked by the host's immune system. Frequency of administration may be determined and adjusted over the course of therapy, and is generally, but not necessarily, based on treatment and/or suppression and/or amelioration and/or delay of a disease. Alternatively, sustained continuous release formulations of a polypeptide may be appropriate. Various formulations and devices for achieving sustained release are known in the art.

In some embodiments, dosage is daily, every other day, every three days, every four days, every five days, or every six days. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen (including the polypeptide used) can vary over time.

In some embodiments, for an adult subject of normal weight, doses ranging from about 0.01 to 1000 mg/kg may be administered. In some embodiments, the dose is between 1 to 200 mg. The particular dosage regimen, i.e., dose, timing and repetition, will depend on the particular subject and that subject's medical history, as well as the properties of the polypeptide (such as the half-life of the polypeptide, and other considerations well known in the art).

For the purpose of the present disclosure, the appropriate dosage of will depend on the specific agent (or compositions thereof) employed, the formulation and route of administration, the type and severity of the disease, whether the polypeptide is administered for preventive or therapeutic purposes, previous therapy, the subject's clinical history and response to the antagonist, and the discretion of the attending physician. Typically the clinician will administer a polypeptide until a dosage is reached that achieves the desired result. Administration of one or more polypeptides can be continuous or intermittent, depending, for example, upon the recipient's physiological condition, whether the purpose of the administration is therapeutic or prophylactic, and other factors known to skilled practitioners. The administration of an agent may be essentially continuous over a preselected period of time or may be in a series of spaced dose, e.g., either before, during, or after developing a disease.

“A subject in need thereof”, refers to an individual who has a disease, a symptom of the disease, or a predisposition toward the disease, with the purpose to cure, heal, alleviate, relieve, alter, remedy, ameliorate, improve, or affect the disease, the symptom of the disease, or the predisposition toward the disease.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In some embodiments, the non-human animal is a mammal (e.g., rodent (e.g., mouse or rat), primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). The non-human animal may be a male or female at any stage of development. The non-human animal may be a transgenic animal or genetically engineered animal.

In some embodiments, the subject is a companion animal (a pet). “A companion animal,” as used herein, refers to pets and other domestic animals. Non-limiting examples of companion animals include dogs and cats; livestock such as horses, cattle, pigs, sheep, goats, and chickens; and other animals such as mice, rats, guinea pigs, and hamsters. In some embodiments, the subject is a research animal. Non-limiting examples of research animals include: rodents (e.g., rats, mice, guinea pigs, and hamsters), rabbits, or non-human primates.

Alleviating a disease includes delaying the development or progression of the disease, or reducing disease severity. Alleviating the disease does not necessarily require curative results. As used therein, “delaying” the development of a disease means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a disease includes initial onset and/or recurrence.

In some embodiments, the disease treated using the engineered cell or composition comprising the engineered cell described herein is cancer. The term “cancer” refers to a class of diseases characterized by the development of abnormal cells that proliferate uncontrollably and have the ability to infiltrate and destroy normal body tissues. See, e.g., Stedman's Medical Dictionary, 25th ed.; Hensyl ed.; Williams & Wilkins: Philadelphia, 1990. Exemplary cancers include, but are not limited to, hematological malignancies. Additional exemplary cancers include, but are not limited to, lung cancer (e.g., bronchogenic carcinoma, small cell lung cancer (SCLC), non-small cell lung cancer (NSCLC), adenocarcinoma of the lung); kidney cancer (e.g., nephroblastoma, a.k.a. Wilms' tumor, renal cell carcinoma); acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); heavy chain disease (e.g., alpha chain disease, gamma chain disease, mu chain disease; hemangioblastoma; hypopharynx cancer; inflammatory myofibroblastic tumors; immunocytic amyloidosis; liver cancer (e.g., hepatocellular cancer (HCC), malignant hepatoma); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva). In some embodiments, the cancer treated using the composition and methods of the present disclosure is melanoma.

In some embodiments, the cancer is a solid tumor. In some embodiments, the cancer is breast cancer. In some embodiments, the cancer is triple negative breast cancer.

In some embodiments, the disease treated using the engineered cell or composition comprising the engineered cell described herein is an autoimmune disease. Non-limiting examples of autoimmune disease include: Multiple Sclerosis, rheumatoid arthritis, inflammatory bowel diseases (IBD), lupus, and ankylosing spondylitis. Some of these disorders are discussed below. In one aspect, the invention provides methods for the treatment of cancer. Still other disorders that can be treated using an FcRn-binding antibody include: scleroderma, Sjogren's syndrome, Goodpasture's syndrome, Wegener's granulomatosis, polymyalgia rheumatica, temporal arteritis/gian cell arteritis, alopecia areata, anklosing spondylitis, antiphospholipid syndrome, autoimmune Addison's disease, autoimmune hemolytic anemia, autoimmune hepatitis, autoimmune inner ear disease, autoimmune lymphoproliferative syndrome (ALPS), autoimmune thrombocytopenic purpura (ATP), Behcet's disease, bullous pemphigoid, cardiomyopathy, celiac sprue-dermatitis, chronic fatigue syndrome immune deficiency syndrome (CFIDS), chronic inflammatory demyelinating polyneuropathy, cicatricial pemphigoid, cold agglutinin disease, CREST Syndrome, Crohn's disease, Dego's disease, dermatomyositis, juvenile dermatomyositis, discoid lupus, essential mixed cryoglobulinemia, fibromyalgia, fibromyositis, Grave's disease, Guillain-Barre syndrome, Hashimoto's thyroiditis, idiopathic pulmonary fibrosis, idiopathic thrombocytopenia purpura (ITP), IgA nephropathy, insulin dependent diabetes (Type I), juvenile arthritis, Meniere's disease, mixed connective tissue disease, myasthenia gravis, pemphigus vulgaris, pemphigus foliaceus, paraneoplastic pemphigus, pernicious anemia, polyarteritis nodosa, polychondritis, polyglancular syndromes, polymyalgia rheumatica, polymyositis, dermatomyositis, primary agammaglobulinemia, primary biliary cirrhosis, psoriasis, Raynaud's phenomenon, Reiter's syndrome, rheumatic fever, sarcoidosis, stiff-man syndrome, Takayasu arteritis, ulcerative colitis, uveitis, vasculitis, vitiligo. In some embodiments, the autoimmune disease is type I diabetes or multiple sclerosis.

Some of the embodiments, advantages, features, and uses of the technology disclosed herein will be more fully understood from the Examples below. The Examples are intended to illustrate some of the benefits of the present disclosure and to describe particular embodiments, but are not intended to exemplify the full scope of the disclosure and, accordingly, do not limit the scope of the disclosure.

### EXAMPLES

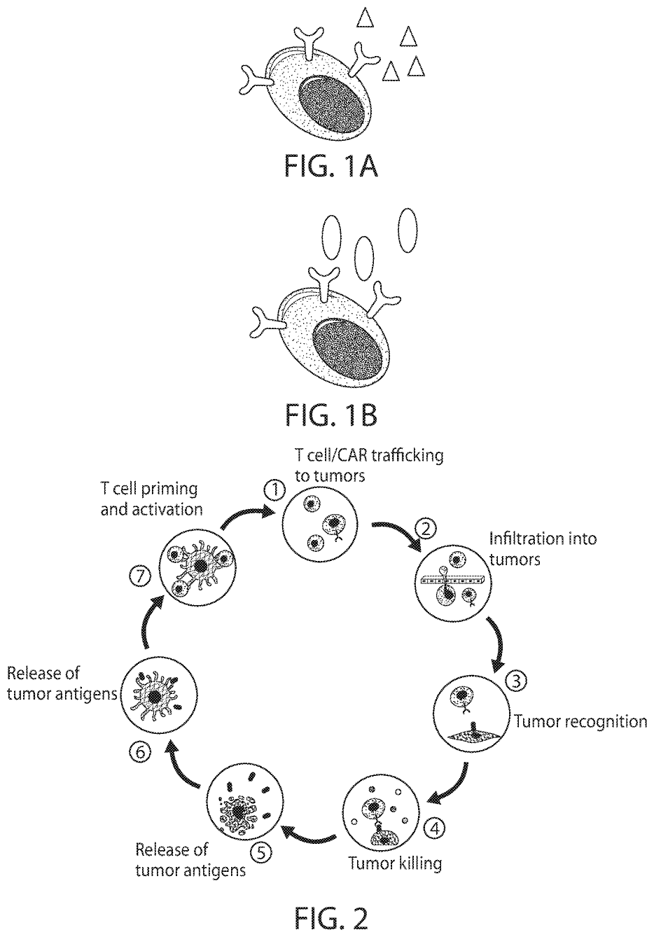

VHH-secreting CAR T cells can be engineered for combination therapies (FIGS. 1A-1B). Cytokine (e.g., IL-12, IL-15, and IL-18) secreting or “armored” CAR T cells have been described. IL-12 secreting CAR T cells are pro-inflammatory and enhance CTLs and NK cells. IL-15 secreting CAR T cells enhance CTLs and NK cells and improve memory. IL-18 secreting CAR T cells support T cell persistence and activity.

The VHH-secreting CAR T cells described herein further increase immune modulation with VHH secretion. VHHs are small and easily packaged in a single vector. They are also stable and can be expressed easily with less metabolic strain. Top candidates engage the innate immune system, as with anti-CD47, and avoid the immunosuppressive environment of the tumor, as with anti-PDL1. Characteristics of successful immunotherapies are illustrated in FIG. 2.

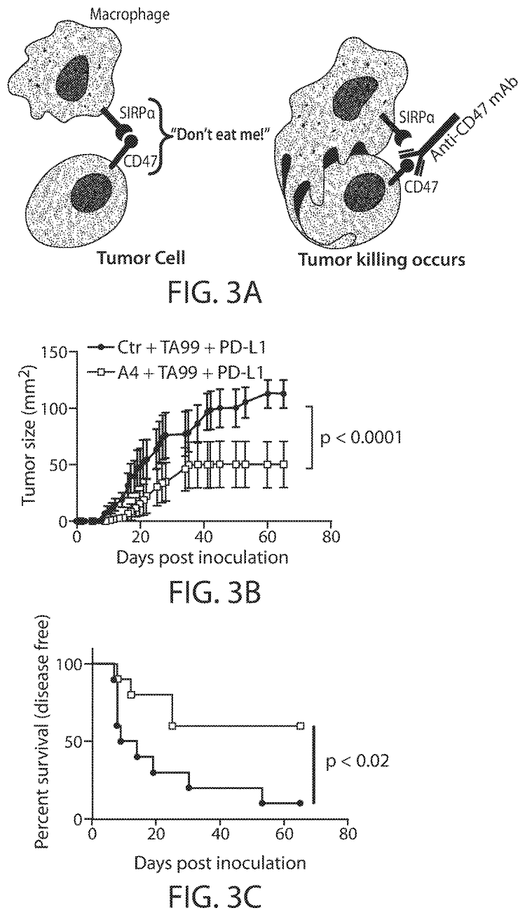

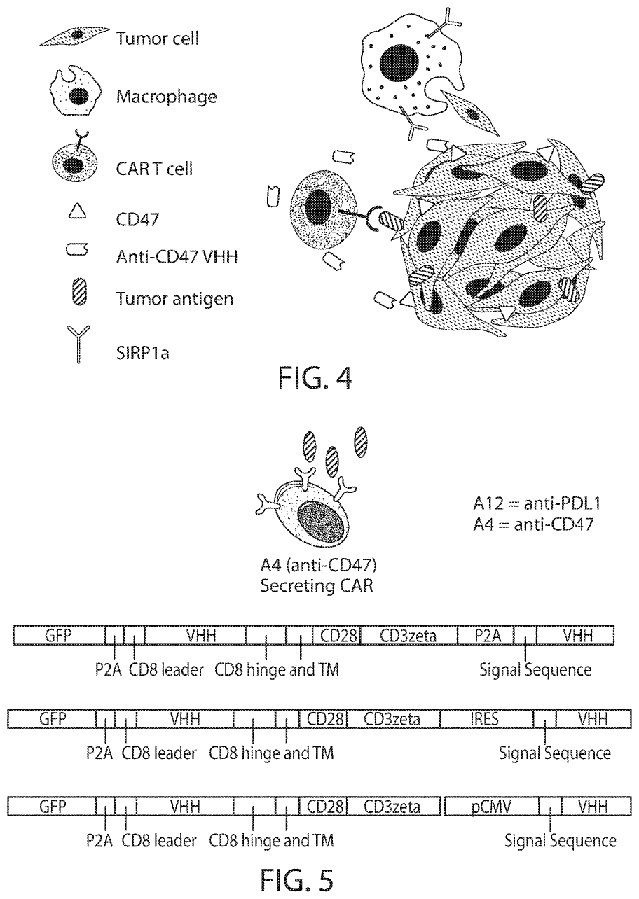

The experiments provided herein sought to determine whether effectiveness could be enhanced by anti-CD47 combination therapy (FIG. 3). CD47 triggers an anti-phagocytic signal and contributes to the immune evasion of cancer cells. It binds to SIRP1a on macrophages and is expressed on a wide range of tumors. Tumor killing efficiency may be enhanced by engaging the innate immune system (FIG. 4). Macrophage inhibition was prevented at the tumor sites by having CARs accumulate in the tumor microenvironment and locally secrete an anti-CD47 VHH to support macrophage phagocytosis.

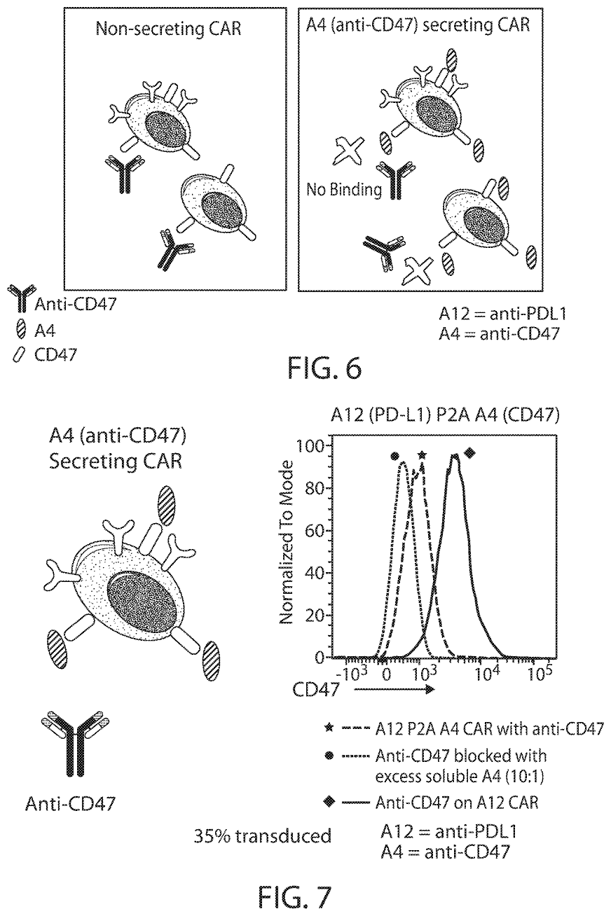

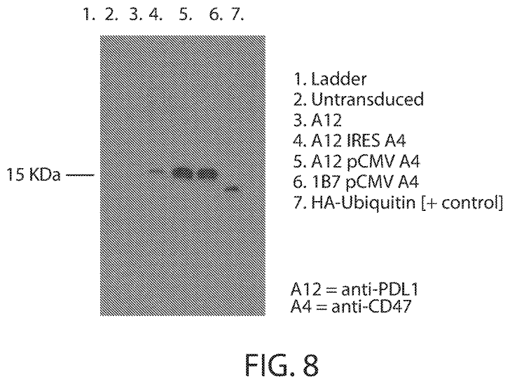

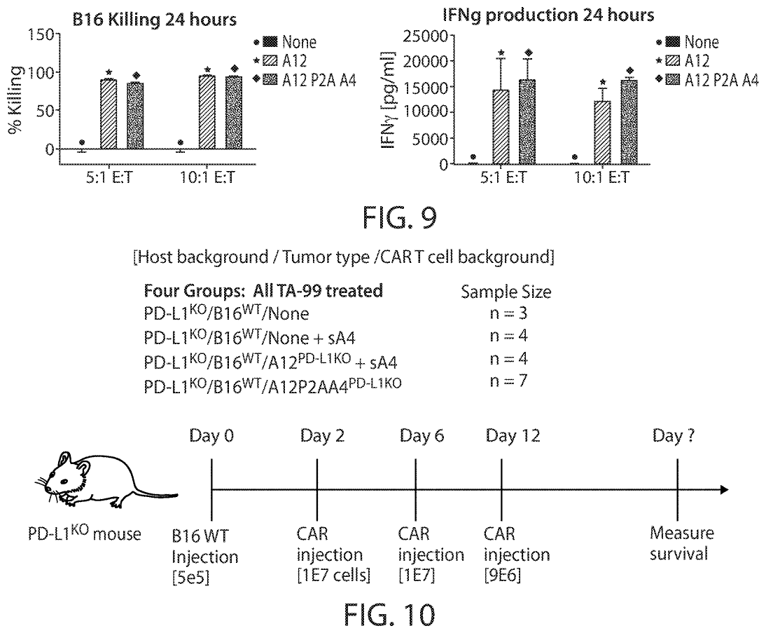

VHH-secreting CARs can be generated for combination therapies (FIG. 5). Three constructs were used to generate a VHH secreting CAR T cell (self-cleaving peptide, internal ribosomal entry site, or two promoter system). All are on a single lentiviral vector. A4 (anti-CD47 VHH) secreting CARs block detection of CD47 (FIG. 6). To show the CAR T cells are secreting the anti-CD47 VHH, a blocking assay was performed where it was expected that the secreted A4 will prevent the fluorescently labeled anti-CD47 mAb from binding to the CD47 on the T cell surface. Both bind competing epitopes. The assay also showed that bystander T cells, which were not transduced with the plasmid, also show CD47 blockade. A12 (PD-L1) P2A (CD47) CARs can secrete functional A4 (FIG. 7). CARs are able to secrete CD47 to sufficiently block the fluorescently labeled anti-CD47 mAb from binding. Anti-HA IP on supernatant or A4-HA secreting CARs is shown in FIG. 8. The secreted CD47 VHH was tagged with an HA tag, and to further show secretion, the presence of the A4 VHH in the supernatant of the CAR T cell cultures was verified via western blot. Engineered cells comprising A12 (anti-PD-L1 VHH) CARs and secreting A4 (anti-CD47 VHH) function in vitro (FIG. 9). To check for tumor killing and CAR T cell activation, the engineered VHH secreting CAR T cells were co-cultured with B16 melanoma cells, and tumor killing was observed. The addition of the A4 VHH secretion does not affect the killing activity of the CAR T cells.

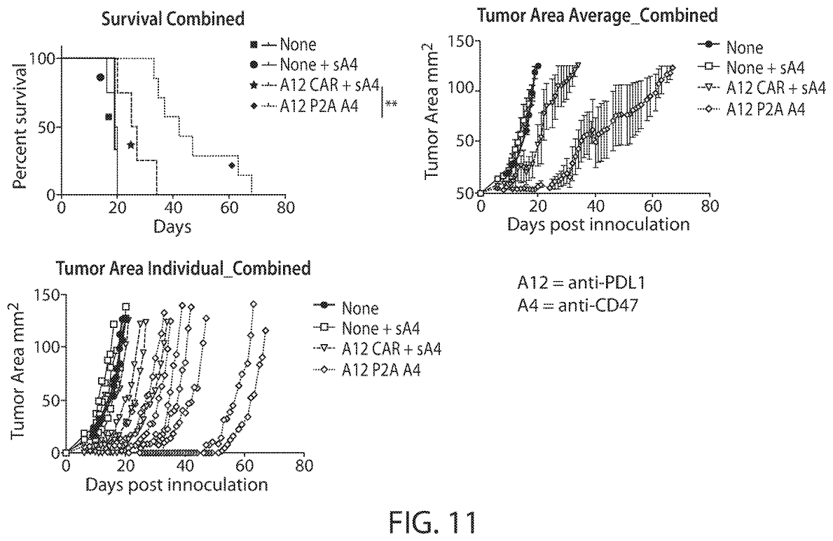

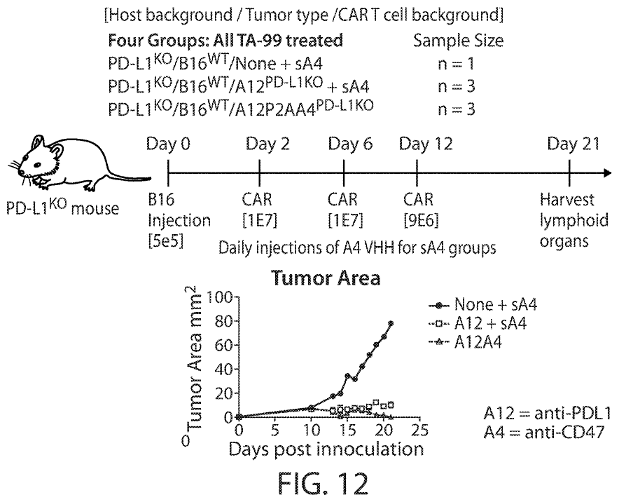

An in vivo experiment on A4 secreting CARs is shown in FIG. 10. The experiment tested whether the localized A4 secretion in PDL1 targeted CARs was beneficial. Mice were either treated with nothing, daily soluble injections of A4 VHH, A12 CAR with daily injections of soluble A4 VHH, or A12 CARs that secrete A4 VHH. Localized A4-secretion improves A12 CAT T cell treatment (FIG. 11). It was observed that localized delivery of the VHH by the CAR T cells provides a survival benefit and decreases the rate of tumor growth in the in vivo, syngeneic model without lymphodepletion. A similar experiment was done to verify that having the excess metabolic strain of producing the A4 VHH did not affect cell persistence, but mice were sacrificed at the mid-point of the experiment to check for the presence of CAR (FIG. 12).

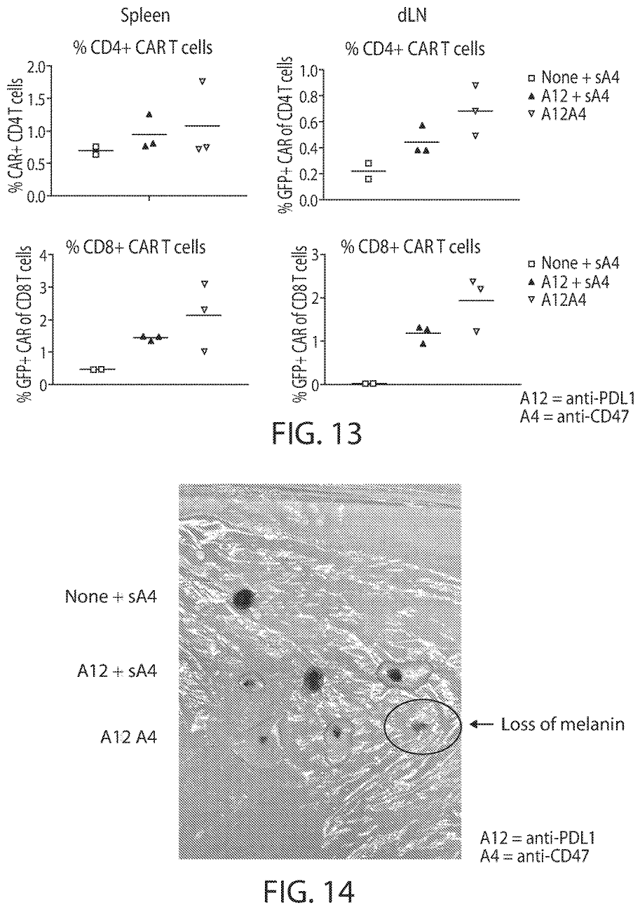

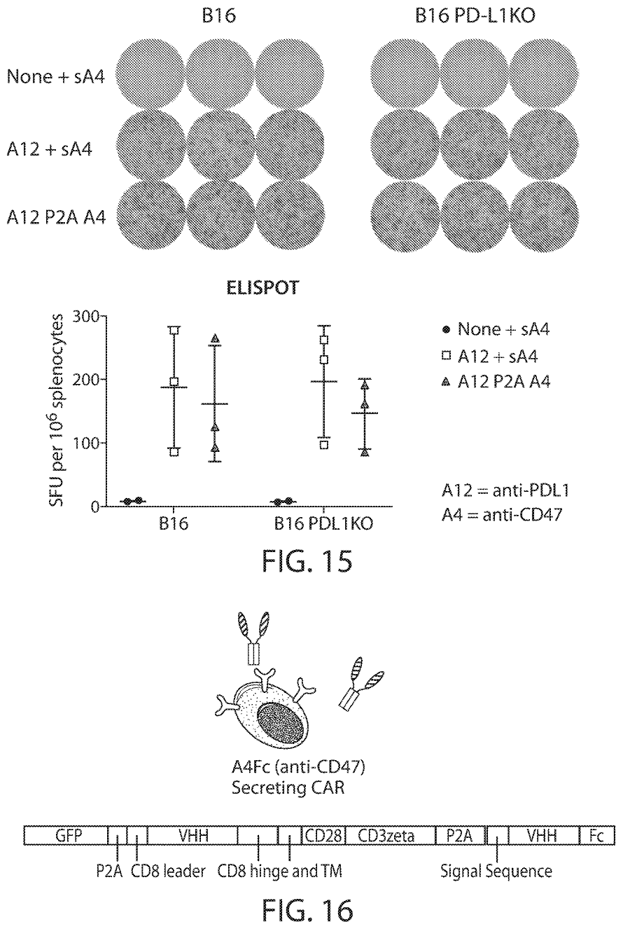

CAR T cell expansion was not negatively affected by A4 secretion (FIG. 13). There was not a negative effect of the A4 secretion on the persistence of the A12 CAR cells. Epitope spreading can be seen with A12A4 treatment. Images of the tumors harvested at day 21 in FIG. 14 show that there was some epitope spreading, as seen by loss of melanin production in one of the tumors treated with the A4 secreting CAR. An ELISPOT assay showed epitope spreading (FIG. 15). The assay performed on the harvested spleens and B16 cells verified the presence of T cells that are reactive against the tumors. Epitope spreading from co-incubation with the B16 PDL1KO cell lines are also seen.

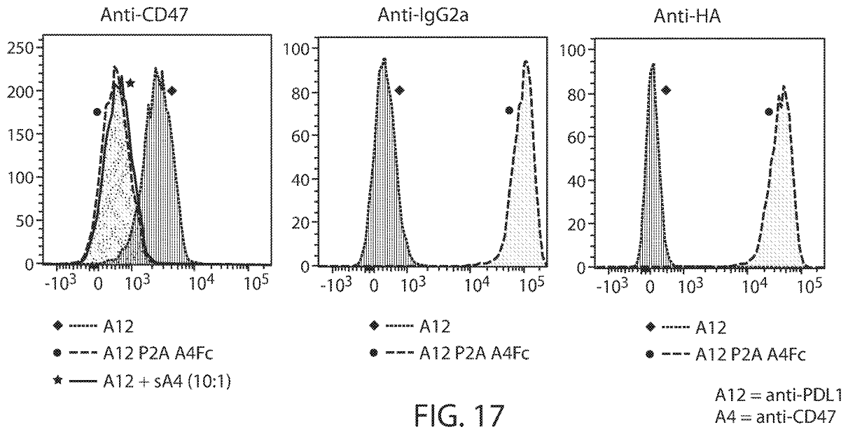

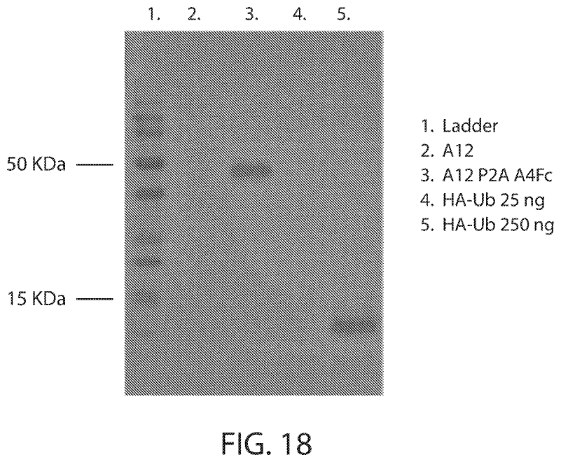

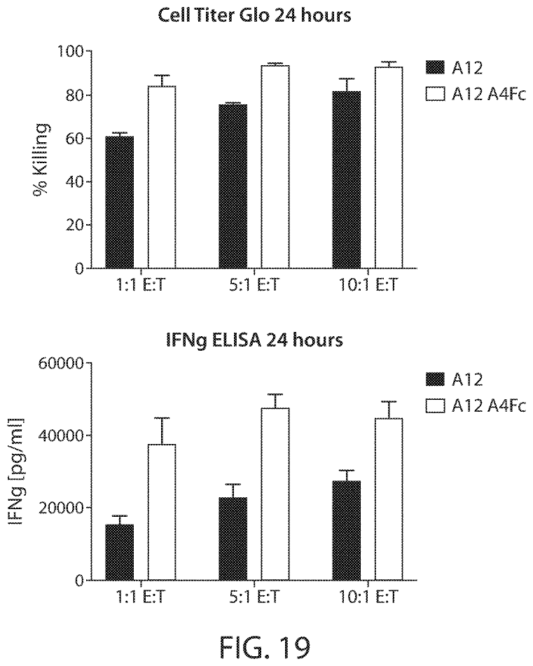

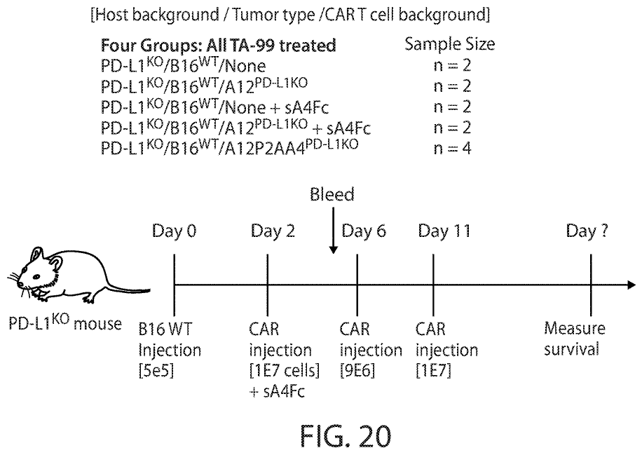

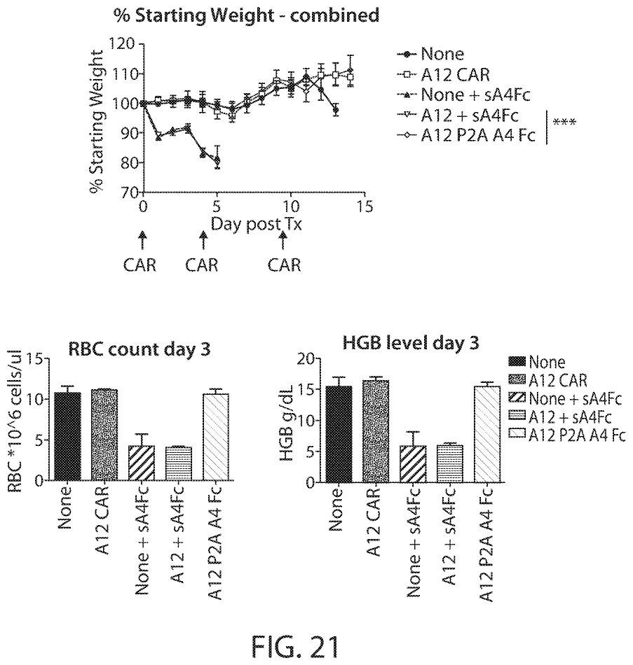

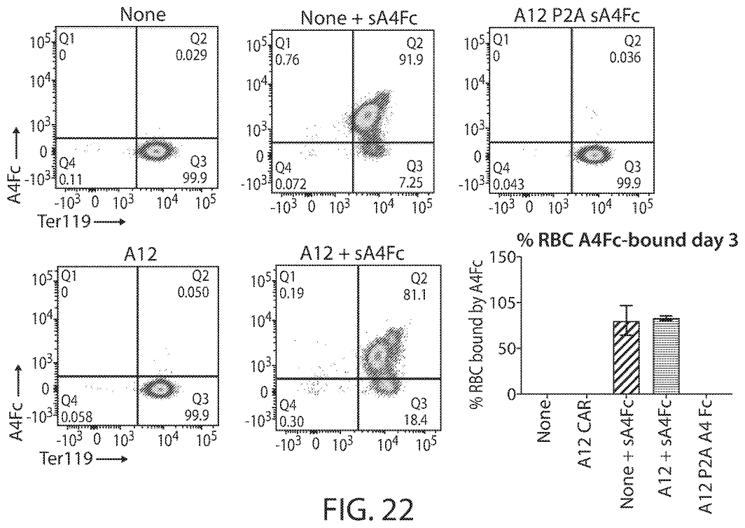

CARs can be used to target delivery of systemically-toxic immune modulators (FIG. 16). VHH-FC fusions can be generated with this construct, providing potential effector function. Engineered cells containing A12 CARs linked to A4Fc via a P2A self-cleaving peptide secreted A4-Fc (FIG. 17). The secreted A4-Fc has an IgG2a Fc domain and an HA tag, so successful secretion of the A4Fc can be proved by a FACs assay. Blocking of endogenous CD47 using a similar CD47 assay was also shown. An IP on the supernatant of the A4Fc secreting CARs showed that the A4Fc was expressed and secreted (FIG. 18). A co-culture assay with B16 cells showed that the secretion of the A4Fc does not negatively impact CAR cell killing (FIG. 19). To show the increased safety profile with localized delivery by CAR T cells, an in vivo assay was set up where mice were inoculated with B16 cells and either untreated, treated with just the A12 CAR, treated with a soluble injection of the A4Fc, the A12 CAR and a soluble injection of the A4Fc, or by a CAR that is secreting the A4Fc (FIG. 20). Targeted A4Fc delivery shows less toxicity (FIG. 21). By measuring mouse weight, it was observed that the soluble dose of A4Fc is toxic and causes weight loss and anemia. However, the localized delivery of A4Fc was safer. Targeted delivery of A4Fc decreases binding to circulating red blood cells (FIG. 22). It was shown that systemic delivery of the A4Fc results heavily in binding of red blood cells, whereas the secreted A4Fc does not heavily impact circulating red blood cells.

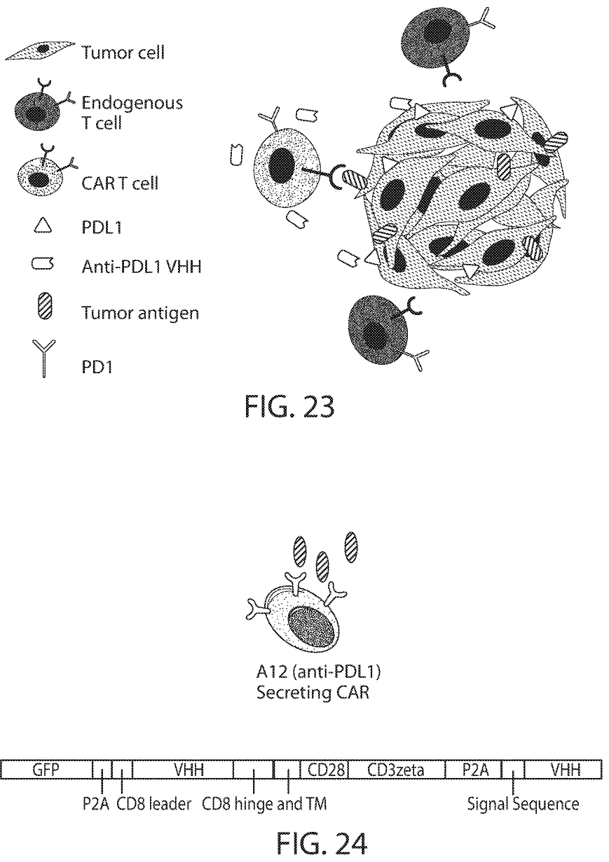

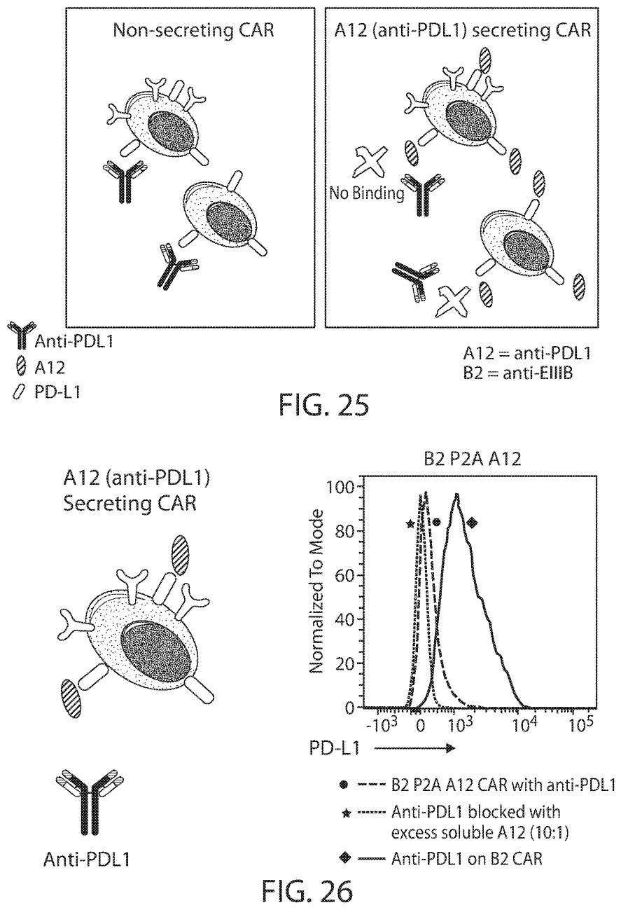

Tumor killing efficiency may be enhanced by preventing T cell exhaustion (FIG. 23). CAR T cells that secrete VHHs that target checkpoint molecules are of interest. They would enhance CAR T cell persistence and activity in the immunosuppressive tumor microenvironment. This may increase T cell activity by endogenous T cells, since tumor checkpoints are blocked. Anti-PDL1-secreting CARs can be generated to decrease T cell exhaustion (FIG. 24). These A12 secreting CARs were generated using a P2A sequence. A12 secreting CARs block detection of PD-L1 (FIG. 25). A blocking assay can be performed, similar to the CD47 assay, to show that the A12 VHH is being successfully secreted, and can bind to the PD-L1 on T cells that are both transduced and untransduced.

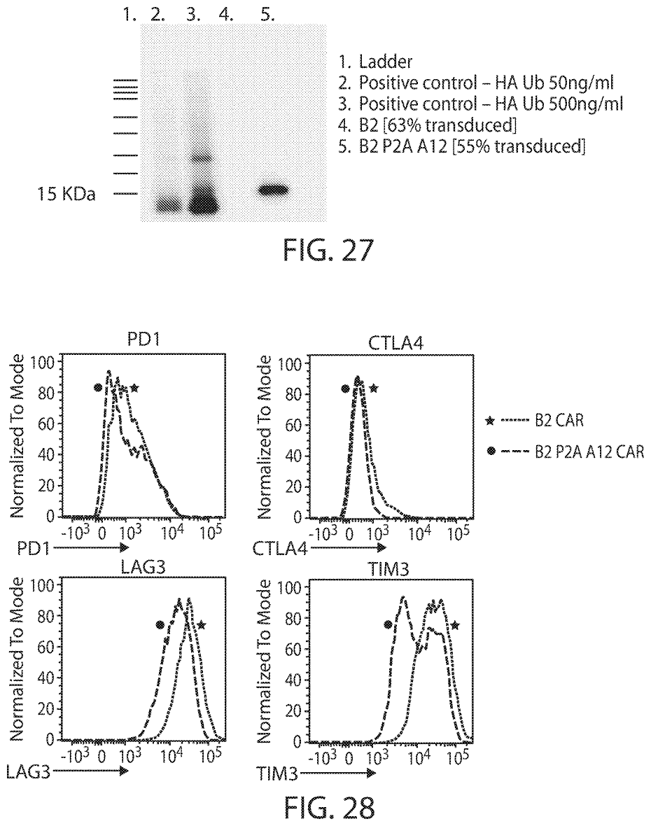

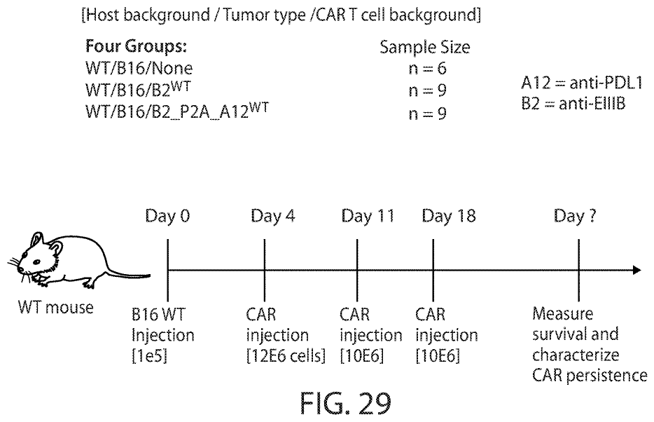

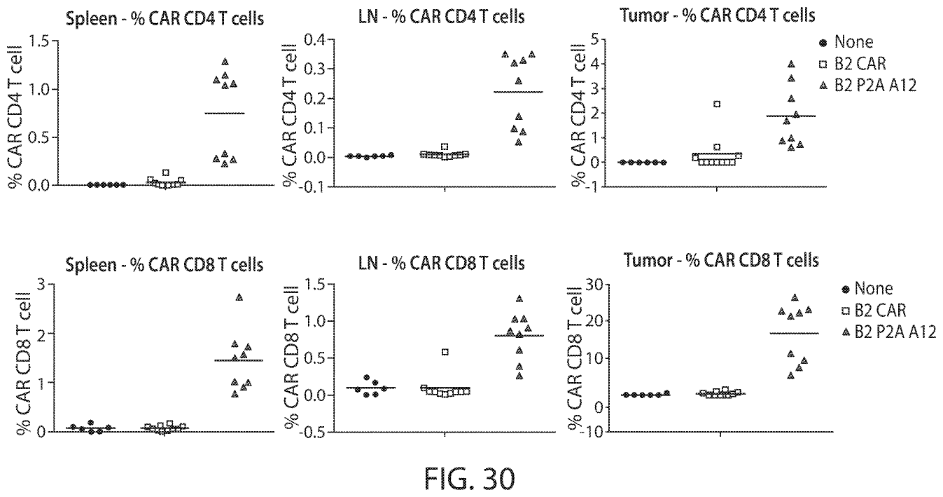

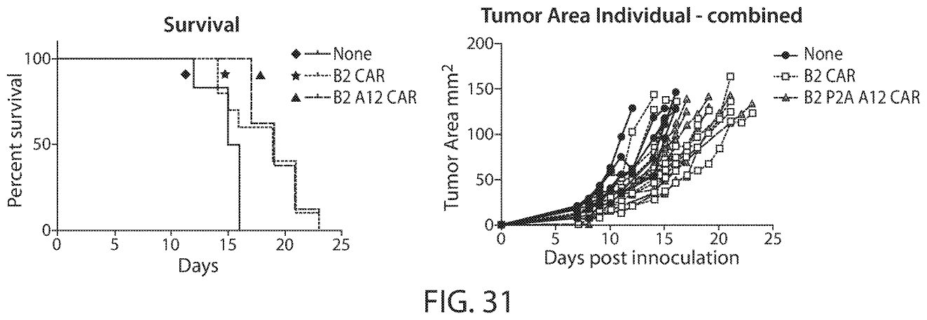

Engineered cells comprising B2 CARs linked to A12 (anti-PD-L1 VHH) via a P2A self-cleavage peptide can secrete functional A12. The results of the FACs binding assay are shown in FIG. 26. Cells in the population where A12 secretion is happening show decreased PDL1 binding by the mAb that binds the same epitope. FIG. 27 shows anti-HA on supernatant of A12-HA secreting CARs. The secreted A12 VHH has an HA tag, and A12 secretion can be verified by harvesting the supernatant of the cultures and showing it is expressed. A12-secreting CARs show less “exhaustion” during generation in vivo (FIG. 28). To verify that CAR T cells show improved persistence, an in vivo experiment was set up where mice were inoculated with B16 tumors and treated with either nothing, B2 CARs (CARs that contain anti-EIIIB fibronectin VHH), or B2 CARs that secrete A12 (FIG. 29). A12 secretion increases persistence of B2 CARs (FIG. 30). Upon harvesting the lymphoid organs and tumors after sacrifice, it was observed that the A12 secreting CAR T cells showed much better persistence than the B2 CARs by themselves. B2 CAR T cells secreting A12 did not significantly increase survival over B2 CART cells alone (FIG. 31).

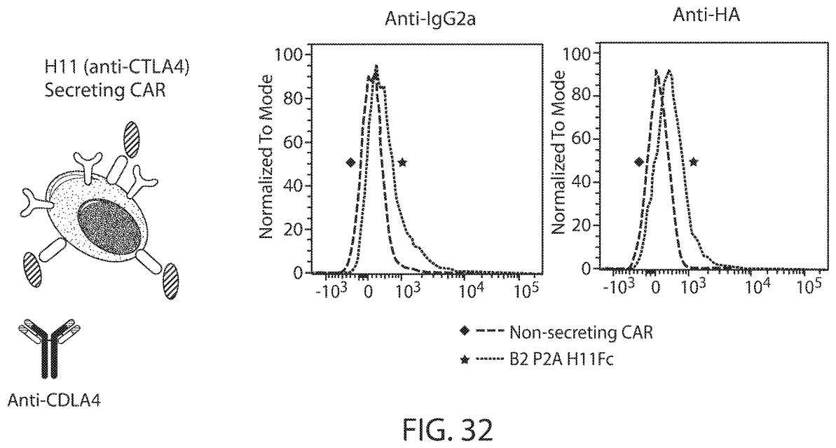

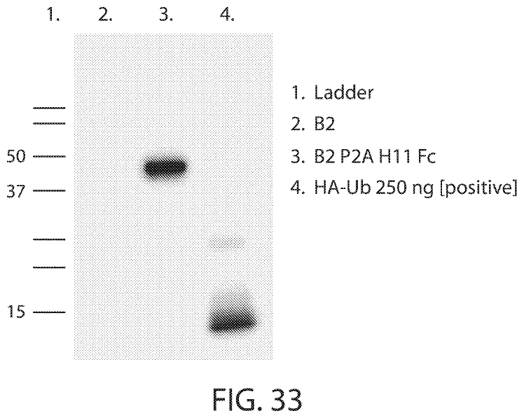

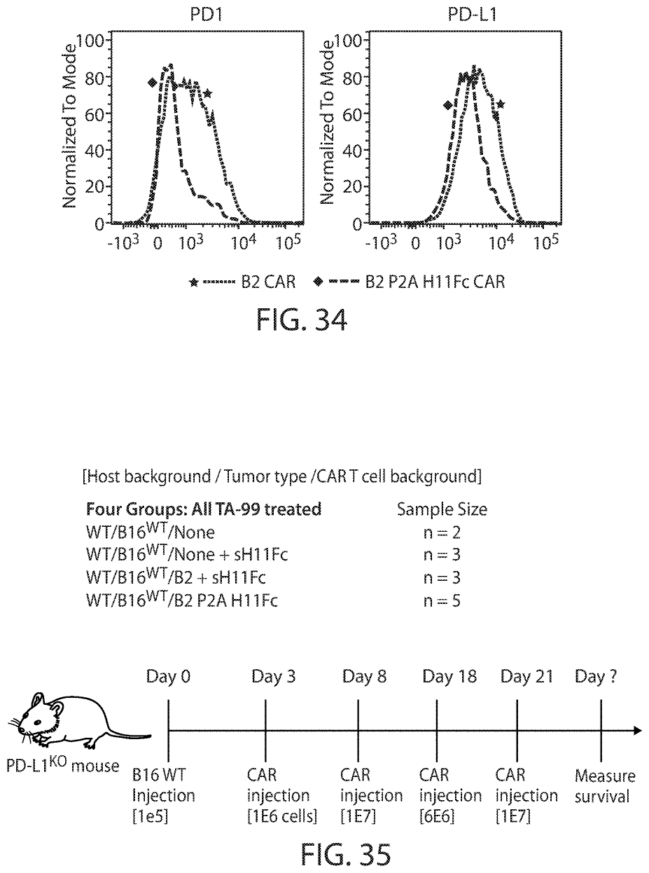

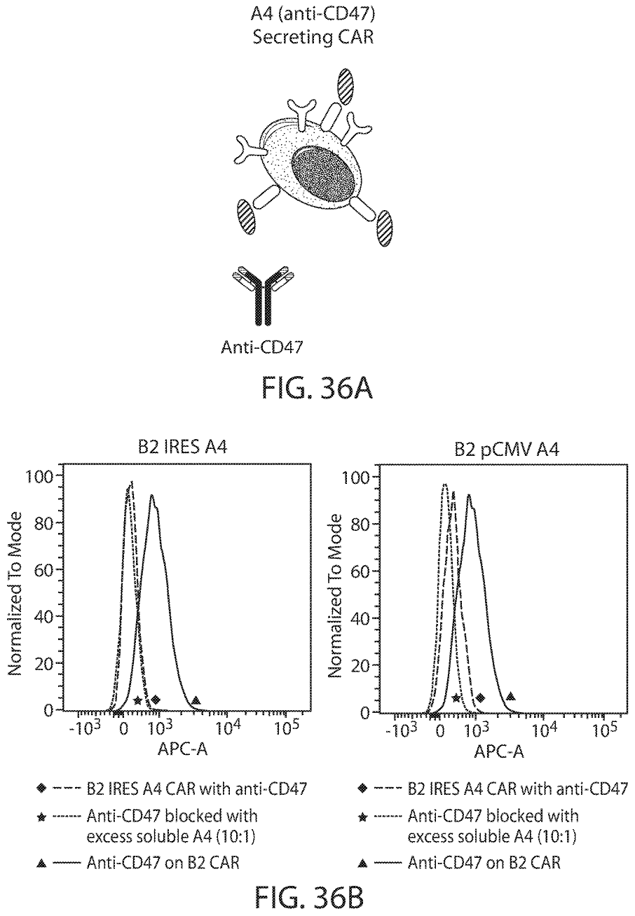

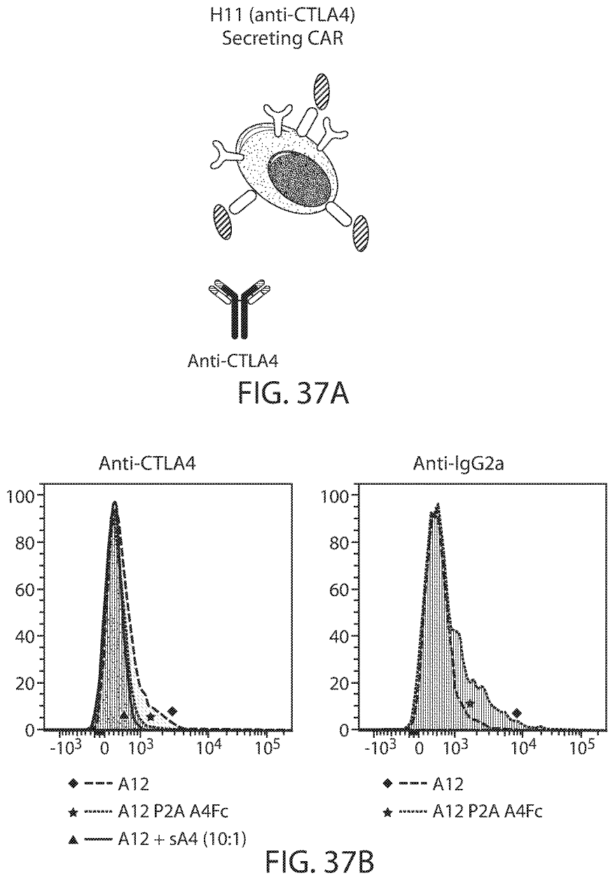

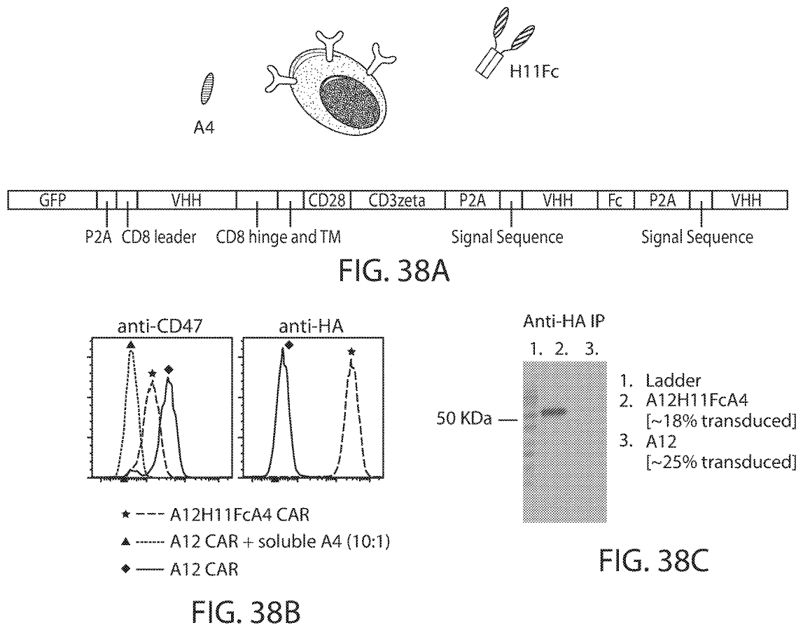

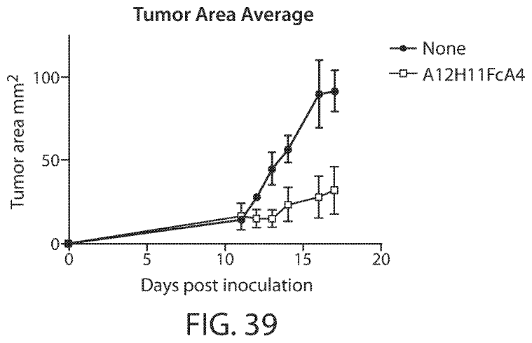

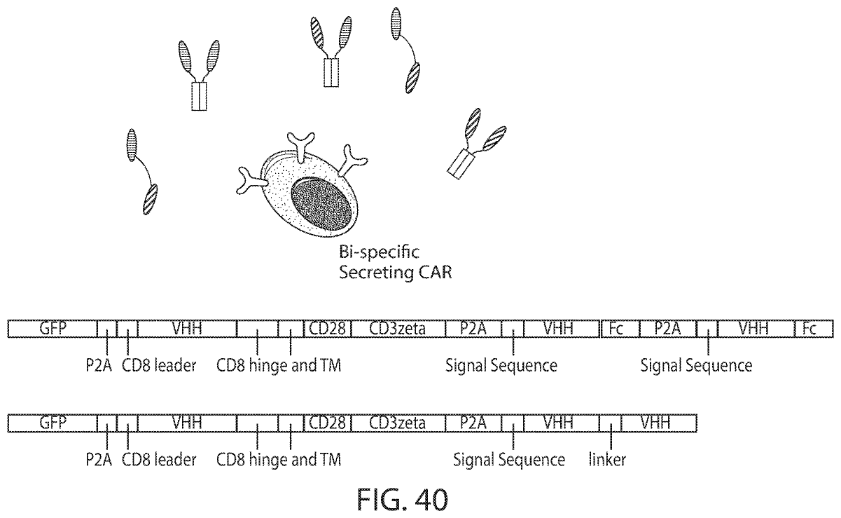

B2 CAR T cells secreting Hi i-Fc (H11 is an anti-CLTA4 VHH) were generated (FIG. 32). CAR T cells that secrete an anti-CTLA4 VHH-Fc fusion were also generated and shown to express the Fc fusion. FIG. 33 shows with IP that the VHH-Fc fusion is expressed. Hi i-Fc secreting CARs show less “exhaustion” during generation in vivo (FIG. 34). FIG. 35 shows ongoing in vivo experiments on Hi i-Fc secreting CARs.

What is claimed is:

1. An engineered cell comprising:
(i) a nucleotide sequence encoding a chimeric antigen receptor (CAR) comprising an extracellular target-binding moiety and an intracellular signaling domain; and
(ii) a nucleotide sequence encoding a heavy-chain antibody (VHH) or a VHH fusion protein thereof.

2. The engineered cell of claim 1, wherein the nucleotide sequence of (i) is operably linked to a first promoter.

3. The engineered cell of claim 1 or 2, wherein the nucleotide sequence of (i) and/or (ii) is operably linked at the 5′ end to a nucleotide sequence encoding a signal sequence.

4. The engineered cell of any one of claims 1-3, wherein (i) and (ii) are linked via a nucleotide sequence encoding a self-cleaving peptide.

5. The engineered cell of claim 4, wherein the self-cleaving peptide is a P2A peptide.

6. The engineered cell of any one of claims 1-3, wherein (i) and (ii) are linked via an internal ribosome entry site (IRES).

7. The engineered cell of any one of claims 1-3, wherein (ii) is operably linked to a second promoter.

8. The engineered cell of any one of claims 1-7, wherein (i) and (ii) are on the same vector.

9. The engineered cell of claim 8, wherein the vector is a lentiviral vector or a retroviral vector.

10. The engineered cell of any one of claims 1-9, wherein the extracellular target-binding moiety of the CAR is an antibody.

11. The engineered cell of claim 10, wherein the antibody is a full-length antibody, an antigen-binding fragment, a single domain antibody, a single-chain variable fragment (scFv), or a diabody.

12. The engineered cell of claim 11, wherein the antibody is a single domain antibody.

13. The engineered cell of claim 12, wherein the single domain antibody is a VHH.

14. The engineered cell of any one of claims 1-13, wherein the extracellular target-binding moiety of the CAR binds a tumor-associated antigen.

15. The engineered cell of claim 14, wherein the tumor associated antigen is selected from the group consisting of: PDL1, EIIIB fibronectin, CEA, PSMA, AXL, HER2, CD133, Muc1, Muc16, Siglec15, and mesothelin.

16. The engineered cell of any one of claims 1-14, wherein the extracellular target-binding moiety of the CAR binds an autoimmune antigen.

17. The engineered cell of claim 16, wherein the autoimmune antigen is selected from the group consisting of: antigen-specific T-cell receptors, B cell receptors, and insulin receptor.

18. The engineered cell of any one of claims 1-17, wherein the nucleotide sequence of (ii) encodes a VHH.

19. The engineered cell of any one of claims 1-17, wherein the nucleotide sequence of (ii) encodes a VHH fusion protein.

20. The engineered cell of claim 19, wherein the VHH fusion protein comprises a VHH fused to a fragment crystallizable region (Fc).

21. The engineered cell of claim 19, wherein the VHH fusion protein comprises a VHH fused to an enzyme, a cytokine, or a different VHH.

22. The engineered cell of any one of claims 1-21, wherein the VHH or VHH fusion protein binds an immune checkpoint protein, a tumor-associated antigen, or an immune cell associated antigen.

23. The engineered cell of any one of claims 1-21, wherein the VHH or VHH fusion protein binds a protein selected from the group consisting of: CD47, CTLA4, PD1, PDL1, TIM3, EIIIB fibronectin, LAG3, VISTA, Siglec15, VEGF, VEGFR, HER2, PSMA, AXL, Muc1, Muc16, MHCI/II.

24. The engineered cell of any one of claims 1-23, wherein the extracellular target-binding moiety of the CAR binds PD-L1 and the VHH or VHH fusion protein binds CD47.

25. The engineered cell of any one of claims 1-23, wherein the extracellular target-binding moiety of the CAR binds PD-L1 and the VHH or VHH fusion protein binds CTLA4.

26. The engineered cell of any one of claims 1-23, wherein the extracellular target-binding moiety of the CAR binds PD-L1 and the VHH or VHH fusion protein binds PD-1.

27. The engineered cell of any one of claims 1-23, wherein the extracellular target-binding moiety of the CAR binds PD-L1 and the VHH or VHH fusion protein binds TIM3.

28. The engineered cell of any one of claims 1-23, wherein the extracellular target-binding moiety of the CAR binds PD-L1 and the VHH or VHH fusion protein binds EIIIB fibronectin.

29. The engineered cell of any one of claims 1-23, wherein the extracellular target-binding moiety of the CAR binds EIIB fibronectin and the VHH or VHH fusion protein binds CD47.

30. The engineered cell of any one of claims 1-23, wherein the extracellular target-binding moiety of the CAR binds EIIB fibronectin and the VHH or VHH fusion protein binds CTLA4.

31. The engineered cell of any one of claims 1-23, wherein the extracellular target-binding moiety of the CAR binds EIIB fibronectin and the VHH or VHH fusion protein binds PD-1.

32. The engineered cell of any one of claims 1-23, wherein the extracellular target-binding moiety of the CAR binds EIIB fibronectin and the VHH or VHH fusion protein binds TIM3.

33. The engineered cell of any one of claims 1-23, wherein the extracellular target-binding moiety of the CAR binds EIIB fibronectin and the VHH or VHH fusion protein binds PD-L1.

34. The engineered cell of any one of claims 1-23, wherein the extracellular target-binding moiety of the CAR binds EIIIB fibronectin and the VHH or VHH fusion protein binds LAG3.

35. The engineered cell of any one of claims 1-23, wherein the extracellular target-binding moiety of the CAR binds EIIIB fibronectin and the VHH or VHH fusion protein binds LAG3 and TIM3.

36. The engineered cell of any one of claims 1-23, wherein the extracellular target-binding moiety of the CAR binds PD-L1 and the VHH or VHH fusion protein binds CD47 and CTLA-4.

37. The engineered cell of any one of claims 1-36, wherein cell is an immune cell.

38. The engineered cell of claim 37, wherein the immune cell is selected from CD4+ T cells, CD8+ T cells, regulatory T cells (Tregs), Natural Killer T (NKT) cells, and Natural Killer (NK) cells.

39. The engineered cell of any one of claims 1-38, wherein the engineered cell secretes the VHH or VHH fusion protein.

40. An engineered cell comprising a chimeric antigen receptor (CAR) comprising an extracellular target-binding moiety and an intracellular signaling domain, wherein the engineered cell secrets a VHH or a VHH fusion protein.

41. A composition comprising the engineered cell of any one of claims 1-40.

42. The composition of claim 41, further comprising a pharmaceutically-acceptable carrier.

43. The engineered cell of any one of claims 1-40, or the composition of claim 41 or claim 42, for use in treating a disease.

44. A method of treating a disease, the method comprising administering to a subject in need thereof a therapeutically effective amount of the engineered cell of any one of claims 1-40, or the composition of claim 41 or claim 42.

45. The method of claim 44, wherein the disease is cancer.

46. The method of claim 45, wherein the disease is autoimmune disease.

47. The method of any one of claims 44-46, wherein the engineered cell or the composition is administered via injection or transfusion.


SEQUENCE LISTING (26 sequences)

SEQ ID NO: 1 - protein, 121 aa, Artificial Sequence
  Synthetic Polypeptide
QVQLVESGGGLVEPGGSLRLSCAASGIIFKINDMGWYRQAPGKRREWVAASTGGDEAIYRDSVKDRFTISRDAKNSVFLQMNSLKPEDTAVYYCTAVISTDRDGTEWRRYWGQGTQVTVSS

SEQ ID NO: 2 - protein, 124 aa, Artificial Sequence
  Synthetic Polypeptide
QVQLVESGGGLVQAGGSLRLSCTASGSTFSRNAMAWFRQAPGKEREFVSGISRTGTNSYDADSVKGRFTISKDNAKNTVTLQMNSLKPEDTAIYYCALSQTASVATTERLYPYWGQGTQVTVSS

SEQ ID NO: 3 - protein, 119 aa, Artificial Sequence
  Synthetic Polypeptide
QVQLVETGGGLVQAGGSLRLSCAASGSTFSHNAGGWYRQAPEKQRELVAGISSDGNINYADSVKDRFTISRDNASNTMYLQMNNLKPEDTAVYVCNIRGSYGNTYYSRWGQGTQVTVSS

SEQ ID NO: 4 - protein, 113 aa, Artificial Sequence
  Synthetic Polypeptide
QVQLQESGGGLAQPGGSLRLSCAASGSTISSVAVGWYRQTPGNQREWVATSSTSSTTATYADSVKGRFTISRDNAKNTIYLQMNSLKPEDTAVYYCKTGLTNWGQGTQVTVSS

SEQ ID NO: 5 - protein, 114 aa, Artificial Sequence
  Synthetic Polypeptide
QVQLQESGGGLVQPGGSLRLSCTASGFTFSMHAMTWYRQAPGKQRELVAVITSHGDRANYTDSVRGRFTISRDNTKNMVYLQMNSLKPEDTAVYYCNVPRYDSWGQGTQVTVSS

SEQ ID NO: 6 - protein, 124 aa, Artificial Sequence
  Synthetic Polypeptide
QVQLVESGGGLVQAGGSLRLSCAASGFTFDDYAIGWFRQAPGKEREGVSCISSSDGSTYYTDSVKGRFTISSDNAKNTVYLQMNSLKPEDTAVYYCAADTTFFGCSLNRDYDYWGQGTQVTVSS

SEQ ID NO: 7 - protein, 123 aa, Artificial Sequence
  Synthetic Polypeptide
QVQLVESGGGMVQPGDSLRLSCVASGRTGSSYIIGWFRQAPGKEREFVARVSPSGGTRDYADSVKGRFTVSRDNAKNTVYLQMDRLKPEDTAIYTCAAAGGKWTADSGEYNYWGQGTQVTVSS

SEQ ID NO: 8 - protein, 128 aa, Artificial Sequence
  Synthetic Polypeptide
QVQLQESGGGLVQAGDSLRLSCAASGRTFSRGVMGWFRRAPGKEREFVAIFSGSSWSGRSTYYSDSVKGRFTISRDNAKNTVYLQMNGLKPEDTAVYYCAAGYPEAYSAYGRESTYDYWGQGTQVTVS

SEQ ID NO: 9 - protein, 115 aa, Artificial Sequence
  Synthetic Polypeptide
QVQLQESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSS

SEQ ID NO: 10 - protein, 123 aa, Artificial Sequence
  Synthetic Polypeptide
QVQLHESGGGLVQPGESLRLSCVASGFTLDHSAVGWFRQVPGKEREKLLCINANGVSLDYADSIKGRFTISRDNAKNTVYLQMNDLKPEDTATYSCAATREFCSAYVFLYEHWGQGTQVTVSS

SEQ ID NO: 11 - DNA, 363 bp, Artificial Sequence
  Synthetic Polynucleotide
caagtccagttggtggagtctggtggtggccttgtggagcctggtggcagcctgcgcctgagctgtgccgccagcgggataattttcaagatcaacgatatgggttggtacagacaggcccccggcaagagacgggaatgggtagccgctagtactggcggtgacgaggctatatatcgcgattctgtgaaggatcggttcactatctcccgcgacgccaaaaattccgtcttcctgcagatgaatagcttgaaacctgaggacacagcggtttactactgtaccgccgtgatttctaccgacagggacggcactgaatggcggcgctactggggccaagggacgcaggtcacggtgtccagc

SEQ ID NO: 12 - DNA, 372 bp, Artificial Sequence
  Synthetic Polynucleotide
caagtgcagcttgtcgaatccggcggcggcctcgtgcaggctggaggcagcctccgattgagctgcactgcttcagggagtaccttctcacggaatgcaatggcctggttcaggcaggcccctggcaaggaacgcgaatttgtctctggtatcagccggacgggtacaaactcctatgatgctgatagtgtaaagggtcggttcacgatttccaaggacaacgcaaaaaacactgtgactcttcaaatgaactcactgaagccggaggacaccgccatatattattgtgccttgagtcagacggccagcgtggccaccacagagcgactctatccctactggggccagggaacacaggtgactgtgtctagt

SEQ ID NO: 13 - DNA, 357 bp, Artificial Sequence
  Synthetic Polynucleotide
caggtgcagctcgtggagactgggggaggcttggtgcaggctggggggtctctgagactctcctgtgcagcctctggaagcacattcagtcataatgccggcggctggtaccgccaggctccagaaaagcagcgcgagttggtcgcagggattagtagtgatggtaacatcaactatgcggactccgtgaaggaccgattcaccatctccagagacaacgccagcaacacgatgtatctacaaatgaacaacctgaaacctgaggacacggccgtctatgtctgtaatatcaggggatcgtacggtaatacctattacagtcggtggggccaggggacccaggtcaccgtctcctca

SEQ ID NO: 14 - DNA, 339 bp, Artificial Sequence
  Synthetic Polynucleotide
caggtgcagctgcaggagtctggaggagggttggcgcagcctggggggtctctgagactctcctgtgcagcctctggaagcacgatcagtagcgtcgccgtgggctggtaccgccagactccagggaatcagcgcgagtgggtcgccactagtagcacgagtagtactaccgcaacgtatgctgactccgtgaagggccgattcaccatctccagagacaacgccaagaacacgatctatctgcaaatgaacagcctgaaacctgaggacacggccgtctattactgtaaaacaggcctgactaattggggccaggggacccaggtcaccgtctcctca

SEQ ID NO: 15 - DNA, 342 bp, Artificial Sequence
  Synthetic Polynucleotide
caggtgcagctgcaggagtcggggggaggcttggtgcagcctggggggtctctgagactttcctgtacagcctctggattcaccttcagtatgcatgccatgacctggtaccgccaggctccagggaagcagcgcgagttggtcgcagttattactagtcatggtgatagggcaaactatacagactccgtgaggggccgattcaccatctccagagacaataccaagaacatggtgtatctgcaaatgaacagcctgaaacctgaggacacggccgtgtattattgtaatgtcccccggtatgactcctggggccaggggacccaggtcaccgtctcctca

SEQ ID NO: 16 - DNA, 372 bp, Artificial Sequence
  Synthetic Polynucleotide
caggtgcagctcgtggagtcggggggaggcttggtgcaggctggggggtctctgagactctcctgtgcagcctctggattcactttcgatgattatgccataggctggttccgccaggccccagggaaggagcgtgagggggtctcatgtattagtagtagtgatggtagcacatactatacagactccgtgaagggccgattcaccatctccagtgacaacgccaagaacacggtgtatctgcaaatgaacagcctgaaacctgaggacacggccgtttattactgtgcagcggacaccactttcttcggctgctctctgaaccgggactatgactactggggccaggggacccaggtcaccgtctcctca

SEQ ID NO: 17 - DNA, 369 bp, Artificial Sequence
  Synthetic Polynucleotide
caggtgcagctcgtggagtcgggtggaggtatggtgcaacctggggactctctgaggctctcctgtgtagcctctggacgcaccggcagtagctatatcataggctggttccgccaggctccaggaaaggagcgtgagtttgtagcgcgtgtttcaccgagcggcggtaccagagactatgcagactccgtgaagggacgattcaccgtctccagagacaacgccaaaaacacggtgtacctgcaaatggacaggctgaaacctgaagacacggccatttatacctgtgctgcggctggggggaaatggacagcggattcgggagagtataactactggggccaggggacccaggtcaccgtctcctca

SEQ ID NO: 18 - DNA, 383 bp, Artificial Sequence
  Synthetic Polynucleotide
caggtgcagctgcaggagtcagggggaggattggtgcaggctggggactctctgagactctcctgcgcagcctctggacgcaccttcagtcgcggtgtaatgggctggttccgccgggctccagggaaggagcgtgagtttgtagcaatctttagcgggagtagctggagtggtcgtagtacatactattcagactccgtaaagggccgattcaccatctccagagacaacgccaagaacacggtgtatctgcaaatgaacggcctgaaacctgaggacacggccgtttattactgtgcagcgggatatccggaggcgtatagcgcctatggtcgggagagtacatatgactactggggccaggggacccaggtcaccgtctc

SEQ ID NO: 19 - DNA, 345 bp, Artificial Sequence
  Synthetic Polynucleotide
caggtgcagctgcaggaatcgggtggtgccctggttcagccgggtggtagcctgcgtctgtcgtgtgctgcgtcgggttttccggttaaccgttatagcatgcgttggtaccgtcaggcaccgggtaaagaacgtgaatgggtcgcgggcatgagctctgccggtgatcgtagttcctatgaagactcagtgaaaggtcgctttaccatttcgcgtgatgacgcacgcaacacggtgtacctgcaaatgaatagtctgaaaccggaagataccgctgtttattactgtaatgttaatgtcggctttgaatactggggtcagggcacgcaggtcaccgtctcctca

SEQ ID NO: 20 - DNA, 369 bp, Artificial Sequence
  Synthetic Polynucleotide
caggtgcagctgcatgagtcagggggaggattggtgcagcctggggagtctctgagactttcctgcgtagcctctggattcactctggatcattctgccgtcggctggttccgccaggtccccgggaaggagcgtgagaaactcttgtgcattaacgctaacggtgttagcctggactatgcagactccattaagggccgattcaccatctctcgggacaacgccaagaacacggtctatctgcagatgaacgacctgaaacctgaggacacagccacatatagctgtgcagcaacgagagaattctgttcagcttatgtgttcctatatgaacactggggccaggggacccaggtcaccgtctcctca

SEQ ID NO: 21 - protein, 225 aa, Artificial Sequence
  Synthetic Polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGPFFLYSKLTVDKSRWQQGNVFSCSVMHEALHNHYTQKSLSLSPGK

SEQ ID NO: 22 - protein, 19 aa, Artificial Sequence
  Synthetic Polypeptide
ATNFSLLKQAGDVEENPGP

SEQ ID NO: 23 - protein, 18 aa, Artificial Sequence
  Synthetic Polypeptide
EGRGSLLTCGDVEENPGP

SEQ ID NO: 24 - protein, 20 aa, Artificial Sequence
  Synthetic Polypeptide
QCTNYALLKLAGDVESNPGP

SEQ ID NO: 25 - protein, 22 aa, Artificial Sequence
  Synthetic Polypeptide
VKQTLNFDLLKLAGDVESNPGP

SEQ ID NO: 26 - DNA, 573 bp, Unknown
  Encephalomyocarditis virus IRES Sequence
cccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcacctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataa